(19) United States
(12) Patent Application Publication
(10) Pub. No.: US 2002/0160975 A1
(43) Pub. Date: Oct. 31, 2002

(54) Conserved XIAP-interaction motif in caspase-9 and Smac/DIABLO for mediating apoptosis

(75) Inventor: Emad S. Alnemri, Ambler, PA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Thomas Jefferson University, Philadelphia, PA (US)

(21) Appl. No.: 10/068,569

(22) Filed: Feb. 6, 2002

Related U.S. Application Data
(63) Continuation-in-part of application No. 09/939,293, filed on Aug. 24, 2001.
(60) Provisional application No. 60/267,966, filed on Feb. 8, 2001.

(51) Int. Cl. A61K 48/00; C07H 21/04

(52) U.S. Cl. 514/44; 536/23.2; 435/69.2; 435/184; 435/320.1; 435/325

(57) ABSTRACT

The invention provides caspase-9-related peptides and polypeptides capable of binding to an Inhibitor of Apoptosis Protein (IAP), as well as caspase-9 mutant that fail to undergo normal processing and fail to bind to an IAP. Nucleic acid molecules, including expression vectors, encoding such peptides and polypeptides are also provided. Such peptides and polypeptides, are useful for inducing apoptosis and identifying inhibitors and enhancer of apoptosis.

### STATEMENT OF GOVERNMENT INTEREST

This invention was made in part with funds provided by the United States Government under National Institutes of Health Research Grants AG14357, AG13487, and CA55227. Accordingly, the United States Government may have certain rights to this invention.

### BACKGROUND OF THE INVENTION

1. Field of the Invention

The present invention relates generally to the regulation of apoptosis, and more particularly, to Inhibitor of Apoptosis Protein binding peptides and polypeptides, and methods of using such polypeptides and peptides to modulate and to identify modulators of apoptosis as well as in therapeutic uses.

2. Description of the Related Art

Apoptosis is a highly conserved cell suicide program essential for development and tissue homeostasis of all metazoan organisms. Changes to the apoptotic pathway that prevent or delay normal cell turnover can be just as important in the pathogenesis of diseases as are abnormalities in the regulation of the cell cycle. Like cell division, which is controlled through complex interactions between cell cycle regulatory proteins, apoptosis is similarly regulated under normal circumstances by the interaction of gene products that either prevent or induce cell death.

Since apoptosis functions in maintaining tissue homeostasis in a range of physiological processes such as embryonic development, immune cell regulation and normal cellular turnover, the dysfunction or loss of regulated apoptosis can lead to a variety of pathological disease states. For example, the loss of apoptosis can lead to the pathological accumulation of self-reactive lymphocytes that occurs with many autoimmune diseases. Inappropriate loss or inhibition of apoptosis can also lead to the accumulation of virally infected cells and of hyperproliferative cells such as neoplastic or tumor cells. Similarly, the inappropriate activation of apoptosis can also contribute to a variety of pathological disease states including, for example, acquired immunodeficiency syndrome (AIDS), neurodegenerative diseases and ischemic injury. Treatments that are specifically designed to modulate the apoptotic pathways in these and other pathological conditions can alter the natural progression of many of these diseases.

Although apoptosis is mediated by diverse signals and complex interactions of cellular gene products, the results of these interactions ultimately feed into a cell death pathway that is evolutionarily conserved between humans and invertebrates. The pathway, itself, is a cascade of proteolytic events analogous to that of the blood coagulation cascade.

Several gene families and products that modulate the apoptotic process have now been identified. Key to the apoptotic program is a family of cysteine proteases termed caspases. The human caspase family includes Ced-3, human ICE (interleukin-1-β converting enzyme) (caspase-1), ICH-1 (caspase-2), CPP32 (caspase-3), ICEᵣₑₗII (caspase-4), ICEᵣₑₗII (caspase-5), Mch2 (caspase-6), ICE-LAP3 (caspase-7), Mch5 (caspase-8), ICE-LAP6 (caspase-9), Mch4 (caspase-10), caspase 11-14 and others.

The caspase proteins share several common features. They are cysteine proteases (named for a cysteine residue in the active site) that cleave their substrates after specific aspartic acid residues (Asp-X). Furthermore, caspases are primarily produced as inactive zymogens, known as procaspases, which require proteolytic cleavage at specific internal aspartate residues for activation. The primary gene product is arranged such that the N-terminal peptide (prodomain) precedes a large subunit domain, which precedes a small subunit domain. The large subunit contains the conserved active site pentapeptide QACXG (X=R, Q, G) (SEQ ID NO:17) which contains the nucleophilic cysteine residue. The small subunit contains residues that bind the Asp carboxylate side chain and others that determine substrate specificity. Cleavage of a caspase yields the two subunits, the large (generally approximately 20 kD) and the small (generally approximately 10 kD) subunit that associate non-covalently: to form a heterodimer, and, in some caspases, an N-terminal peptide of varying length. The heterodimer may combine non-covalently to form a tetramer.

Caspase zymogens are themselves substrates for caspases. Inspection of the interdomain linkages in each zymogen reveals target sites (i.e. protease sites) that indicate a hierarchical relationship of caspase activation. By analyzing such pathways, it has been demonstrated that caspases are required for apoptosis to occur. Moreover, caspases appear to be necessary for the accurate and limited proteolytic events that are the hallmark of classic apoptosis (see Salvesen and Dixit, Cell 91:443-446, 1997). During apoptosis, the initiator caspase zymogens are activated by autocatalytic cleavage, which then activate the effector caspases by cleaving their inactive zymogens (Salvesen and Dixit, Proc. Natl. Acad. Sci. USA 96:10964-10967, 1999; Srinivasula et al., Mol. Cell. 1:949-957, 1998). This characteristic indicates that caspases implicated in apoptosis may execute the apoptotic program through a cascade of sequential activation of initiators and effector procaspases (Salvesen and Dixit, Cell 91:443-446, 1997). The initiators are responsible for processing and activation of the effectors. The effectors are responsible for proteolytic cleavage of a number of cellular proteins leading to the characteristic morphological changes and DNA fragmentation that are often associated with apoptosis (reviewed in Cohen, Biochem. J. 326:1-16, 1997; Henkart, Immunity 4:195-201, 1996; Martin and Green, Cell 82:349-352, 1995; Nicholson and Thomberry, TIBS 257:299-306, 1997; Porter et al., BioEssays 19:501-507, 1997; Salvesen and Dixit, Cell 91:443-446, 1997). The first evidence for an apoptotic caspase cascade was obtained from studies on death receptor signaling (reviewed in Fraser and Evan, Cell 85:781-784, 1996; Nagata, Cell 88:355-365, 1997) which indicated that the death signal is transmitted in part by sequential activation of the initiator procaspase-8 and the effector procaspase-3 (Boldin et al., Cell 85:803-815, 1996; Femandes-Alnernri et al., Proc. Natl. Acad. Sci. USA 93:7464-7469, 1996; Muzio et al., Cell 85:817-827, 1996; Srinivasula et al., Proc. Natl. Acad. Sci. USA 93:13706-13711, 1996). More direct evidence was provided when it was demonstrated that the cytochrome c death signal is transmitted through activation of a cascade involving procaspase-9 and caspase-3 (Li et al., Cell 91:479-489, 1997).

The initiator caspase zymogens are activated by adaptor proteins such as FADD and Apaf-1, which associate in a stimulus-dependent manner with the prodomains of these zymogens and promote their activation via oligomerization (Salvesen and Dixit, Proc. Natl. Acad. Sci. USA 96:10964-10967, 1999; Srinivasula et al., Mol. Cell. 1:949-957, 1998). For example, ligands binding to the cell surface death receptors triggers binding of procaspase-8 to FADD and its subsequent activation and release from the death receptor complex. Likewise, release of cytochrome c from the mitochondria in response to apoptotic stimuli such as serum starvation, ionization radiation, DNA damaging agents etc. triggers oligomerization of Apaf-1 in an ATP or dATP dependent manner. The oligomeric Apaf-1 apoptosome then recruits and activates procaspase-9.

Given the potentially irreversible caspase cascade triggered by activation of the upstream initiator caspases, it is crucial that activation of caspases in the cell be tightly regulated. A number of cellular proteins have been shown to modulate caspase activation and activity. One of these, FLAME/FLIP, inhibits death receptor-mediated activation of caspase-8 by binding to FADD (Irmler et al., Nature 388:190-195, 1997; Srinivasula et al., J. Biol. Chem. 272:18542-18545, 1997). Others, such as the anti-apoptotic members of the Bcl-2 family, inhibit Apaf-1-mediated activation of caspase-9 by blocking cytochrome c release from the mitochondria (reviewed in Adams and Cory, Science 281:1322-1326, 1998; Green and Reed, Science 281:1309-1312, 1998). Heat shock proteins, Hsp70 and Hsp90, also interfere with the mitochondrial apoptotic pathway by modulating the formation of a functional Apaf-1 apoptosome (Saleh, et al., Nature Cell. Biol. 2:476-483, 2000: Pandey, et al., EMBO J. 19:4310-4322, 2000). Finally, members of the Inhibitor of Apoptosis Protein (IAP) family, such as XIAP, c-IAP-1, and c-IAP-2, block both the death receptor and mitochondrial pathways by inhibiting the activity of the effector caspase-3 and caspase-7 and the initiator caspase-9 (reviewed in Deveraux and Reed, Genes Dev. 13:239-252, 1999).

Smac/DIABLO, a mitochondrial protein, which is released together with cytochrome c from the mitochondria in response to apoptotic stimuli, was found to promote caspase activation by binding and neutralizing the IAPs (Du et al., Cell 102:33-42, 2000; Verhagen et al., Cell 102:43-53, 2000).

Accordingly, as IAP, caspase-9, and Smac all play key roles in regulating apoptosis, there exists a need in the art to identify key interactions between these proteins as well as modulators of the same. The present invention relates to this and other advantages related to the newly identified interaction motif.

### SUMMARY OF THE INVENTION

In a first aspect of the invention, the present invention provides an isolated nucleic acid molecule comprising a polynucleotide having a sequence encoding a peptide or polypeptide comprising at least a consensus IAP-binding motif amino acid sequence, as set forth in SEQ ID NO:13, wherein said peptide or polypeptide specifically binds to at least a portion of an Inhibitor of Apoptosis Protein (IAP). In certain embodiments, the encoded peptide or polypeptide binds to at least a portion of an IAP. In specific embodiments, the portion of an IAP is at least one BIR domain, and the BIR domain may be BIR1, BIR2, or BIR3. In other embodiments, the peptide or polypeptide specifically binds to a full-length IAP.

In another aspect of the present invention, nucleic acids of the invention comprise polynucleotides encoding a peptide or polypeptide that contains an amino acid sequence corresponding to the first four residues of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In yet another aspect, the invention provides a nucleic acid molecule consisting essentially of a polynucleotide encoding a peptide or polypeptide including at least an N-terminus amino acid sequence corresponding to a caspase-9 linker, as set forth in SEQ IDNO:11.

In another aspect, the invention includes an isolated nucleic acid molecule consisting essentially of a polynucleotide having a sequence encoding a peptide or polypeptide comprising at least an N-terminus amino acid sequence of Ala-Val-Pro-Tyr, as set forth in SEQ ID NO:15.

In a related aspect, the invention provides an isolated nucleic acid molecule consisting essentially of a polynucleotide having a sequence encoding a peptide or polypeptide comprising at least an N-terminus amino acid sequence corresponding to a Smac N7 peptide, as set forth in SEQ ID NO:12.

In another aspect of the invention, the present invention provides an isolated nucleic acid molecule comprising a polynucleotide encoding a peptide or polypeptide containing a portion of a procaspase-9 that specifically binds at least a portion of an IAP and a portion of a procaspase-9 containing a mutated active site, wherein said peptide or polypeptide specifically binds at least a portion of an IAP and lacks cysteine protease activity.

In a further aspect of the invention, the invention provides an isolated nucleic acid molecule containing a polynucleotide encoding a peptide or polypeptide that includes a consensus IAP-binding motif amino acid sequence, as set forth in SEQ ID NO:13, and at least a portion of a caspase-3, wherein the peptide or polypeptide exhibits caspase-3 enzymatic activity that is inhibited by at least a portion of an IAP. In certain embodiments, the enzymatic activity is inhibited by a full-length IAP. In some embodiments, the encoded peptide or polypeptide consists essentially of a caspase-3 in which the amino acid residues corresponding to the amino-terminal two residues of the p12 subunit are substituted with Ala-Val. In other embodiments, the encoded peptide or polypeptide consists essentially of a caspase-3 in which the amino acid residues corresponding to the amino-terminal four residues of the p12 subunit are substituted with a consensus IAP-binding domain sequence, as set forth in SEQ ID NO:13.

In one aspect, the invention provides an isolated nucleic acid molecule comprising a polynucleotide encoding a peptide or polypeptide containing at least a portion of a mutated procaspase-9, wherein said portion fails to undergo normal processing and possesses wild type caspase-9 enzymatic activity. In one embodiment, the polynucleotide contains any mutation that prohibits cleavage of the encoded polypeptide at a normal cleavage site. In specific embodiments, the portion of mutated procaspase-9 corresponds to human caspase-9 (SEQ ID NO:1) with one or more of amino acids 306, 315, and 330 mutated or substituted by another amino acid. In one specific embodiment, the portion of mutated procaspase-9 corresponds to human caspase-9 with amino acid residue 315 mutated. In other embodiments, the portion of mutated procaspase-9 corresponds to human caspase-9 with amino acid residues 315 and 330 mutated. In yet another embodiment, the portion of mutated procaspase-9 corresponds to human caspase-9 with amino acid residues 306, 315, and 330 mutated. In specific embodiments, mutations of amino acid residues 306, 315, or 330 are Ala substitutions. In further embodiments, the portion of mutated procaspase-9 corresponds to SEQ ID NO:1 with amino acid residues 316 through 330 deleted.

In another aspect of the invention, the invention provides an expression vector containing a nucleic acid molecule of the invention, operatively linked to regulatory elements. In certain embodiments, the regulatory elements include an inducible promoter.

In a related aspect of the invention, the invention provides a host cell containing an expression vector of the invention. In certain embodiments, the cell is a bacterium, a yeast, an animal cell, or a plant cell.

In one aspect of the invention, the present invention provides a peptide or polypeptide containing at least a consensus IAP-binding motif amino acid sequence, as set forth in SEQ ID NO:13, wherein the peptide or polypeptide specifically binds to at least a portion of an Inhibitor of Apoptosis Protein (IAP). In certain embodiments, this portion is at least one BIR domain. In a specific embodiment, this BIR domain is BIR3. In other embodiments, the peptide or polypeptide specifically binds to a full-length IAP.

In another aspect of the present invention, peptides or polypeptides of the invention contain an amino acid sequence corresponding to the first four residues of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In yet another aspect, the invention provides a peptide or polypeptide including at least an N-terminus amino acid sequence corresponding to a caspase-9 linker, as set forth in SEQ ID NO:11, or a variant thereof.

In another aspect, the invention includes a peptide or polypeptide containing at least an N-terminus amino acid sequence of Ala-Val-Pro-Tyr, as set forth in SEQ ID NO:15, or a variant thereof.

In yet another aspect of the invention, the invention provides a peptide or polypeptide includes a caspase-9 linker peptide, as set forth in SEQ ID NO:15, or a variant thereof, wherein the peptide or polypeptide specifically binds to at least a portion of an IAP.

In a further aspect of the invention, the invention provides a peptide or polypeptide comprising the Smac N7 peptide amino acid residues set forth in SEQ ID NO:12, or a variant thereof, wherein the peptide or polypeptide specifically binds to at least a portion of an IAP.

In another aspect, the invention provides a peptide or polypeptide containing a portion of a procaspase-9, or a variant thereof, that specifically binds to at least a portion of an IAP and a portion of a procaspase-9, or a variant thereof, containing a mutated active site, wherein the peptide or polypeptide specifically binds to at least a portion of an IAP and lacks cysteine protease activity.

In yet another aspect, the invention provides a peptide or polypeptide comprising an amino acid sequence of SEQ ID NO:13, and further comprising at least a portion of a caspase-3, or a variant thereof, wherein the peptide or polypeptide exhibits caspase-3 enzymatic activity that is inhibited by an IAP BIR3 domain. In certain embodiments, the amino-terminal two residues of the p12 subunit of caspase-3, or a variant thereof, are substituted with Ala-Val. In another embodiment, the amino-terminal four residues of the p12 subunit of caspase-3, or a variant thereof, are substituted with any four contiguous residues set forth in SEQ ID NO:13.

In yet another aspect, the invention provides a peptide or polypeptide comprising at least a portion of a mutated procaspase-9, or a variant thereof, wherein the portion fails to undergo normal processing and possesses wild type caspase-9 enzymatic activity. In specific embodiments, the portion of mutated procaspase-9 corresponds to human caspase-9 (SEQ ID NO:1) with amino acid residue 315 substituted by Ala. In other embodiments, the portion of mutated procaspase-9 corresponds to human caspase-9 with amino acid residues 315 and 330 substituted by Ala. In yet other embodiments, the portion of mutated procaspase-9 corresponds to human caspase-9 with amino acid residues 306, 315, and 330 substituted by Ala. In further embodiments, the portion of mutated procaspase-9 corresponds to SEQ ID NO:1 with amino acid residues 316 through 330 deleted.

In one aspect of the invention, the present invention provide antibodies that that specifically bind to a peptide or polypeptide with a consensus IAP-binding motif, as set forth in SEQ ID NO:13, that specifically binds to at least a portion of an IAP. In certain embodiments, these antibodies are capable of inhibiting the binding of said peptide or polypeptide to the portion of an IAP normally bound. In specific embodiments, the portion of an IAP bound is at least one BIR domain, and this BIR domain may be BIR1, BIR2, or BIR3. In other embodiments, the antibody will be capable of inhibiting binding of the peptide or polypeptide to a full-length IAP.

In another aspect of the invention, the invention provides an antibody that specifically binds to an epitope located on the N-terminus of a caspase-9-p12 subunit. In certain embodiments, the antibody inhibits the binding of a caspase-9-p12 to at least a portion of an IAP. In specific embodiments, the portion of an IAP bound is at least one BIR domain, and this BIR domain may be BIR1, BIR2, or BIR3. In other embodiments, the antibody will inhibit binding of the peptide or polypeptide to a full-length IAP.

In other aspects, the invention provides a method for inducing apoptosis in a cell comprising contacting the cell with a peptide, polypeptide, nucleic acid, or antibody of the invention, under conditions and for a time sufficient to permit the induction of apoptosis in the cell. In certain aspects of this method, the peptide or polypeptide is capable of inhibiting caspase-9-p12 binding to at least a portion of an IAP. In specific embodiments, the portion is at least one BIR domain. In specific embodiments, the BIR domain is BIR1, BIR2, or BIR3. In other aspects of the method, the polypeptide is a procaspase-9 mutant that fails to undergo normal processing. In a related aspect, the polypeptide is a procaspase-9 mutant that fails to undergo normal processing. In certain embodiments, the cell overexpresses a peptide or polypeptide capable of inhibiting IAP binding to caspase-9.

In another aspect of the invention, the invention provides a method of stimulating apoptosis in a neoplastic or tumor cell, comprising contacting the cell with a nucleic acid, peptide, polypeptide, or antibody of the invention, under conditions and for a time sufficient to permit the induction of apoptosis in the cell. In one aspect of the method, the peptide or polypeptide is capable of inhibiting caspase-9-p12 binding to at least a portion of an IAP. In another aspect, the peptide or polypeptide is a procaspase-9 mutant that fails to undergo normal processing. In some embodiments, the cell overexpresses a peptide of polypeptide capable of inhibiting caspase-9-p12 binding to at least a portion of an LAP. In one embodiment, the cell overexpresses a procaspase-9 mutant that fails to undergo normal processing. In another embodiment, the cell overexpresses an inhibitor of a caspase. In specific embodiments, the inhibitor inhibits activation or activity of caspase-9. In some embodiments, the inhibitor is at least a portion of an Inhibitor of Apoptosis protein.

In yet another aspect of the invention, the present invention provides a method of identifying an inhibitor or enhancer of a caspase-mediated apoptosis comprising contacting a cell containing a vector expressing a peptide or polypeptide containing a consensus IAP-binding motif, as set forth in SEQ ID NO:13, that is capable of specifically binding to at least a portion of an IAP with a candidate inhibitor or candidate enhancer and detecting cell viability, wherein an increase in cell viability as compared to a control indicates the presence of an inhibitor and a decrease in cell viability as compared to a control indicates the presence of an enhancer.

In another aspect of the invention, the present invention provides a method of identifying an inhibitor or enhancer of a caspase-mediated apoptosis comprising contacting a cell containing a vector expressing a peptide or polypeptide containing the first four residues of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10 and detecting cell viability, wherein an increase in cell viability as compared to a control indicates the presence of an inhibitor and a decrease in cell viability as compared to a control indicates the presence of an enhancer.

In a further aspect of the invention, the present invention provides a method of identifying an inhibitor or enhancer of a caspase-mediated apoptosis comprising contacting a cell containing a vector expressing a peptide or polypeptide comprising at least a consensus IAP-binding motif amino acid sequence, as set forth in SEQ ID NO:13, that is capable of specifically binding to at least a portion of an IAP with a candidate inhibitor or candidate enhancer and detecting the presence of large and small caspase subunits, and therefrom determining the level of caspase processing activity, wherein a decrease in processing as compared to a control indicates the presence of an inhibitor and an increase in processing indicates the presence of an enhancer. In certain embodiments of this method, the caspase detected is caspase-3, caspase-7, or caspase-9.

In another aspect of the invention, the present invention provides a method of identifying an inhibitor or enhancer of a caspase-mediated apoptosis comprising contacting a cell containing a vector expressing a polypeptide of the invention, detecting the presence of large and small caspase subunits, and therefrom determining the level of caspase processing activity, wherein a decrease in processing as compared to a control indicates the presence of an inhibitor and an increase in processing indicates the presence of an enhancer. In certain embodiments of the method, the caspase detected is caspase-3, caspase-7, or caspase-9.

In one aspect of the invention, the present invention provides a method of identifying an inhibitor or enhancer of a caspase-mediated apoptosis comprising contacting a cell containing a vector expressing a peptide or polypeptide comprising at least an amino acid sequence corresponding to the consensus IAP-binding motif, as set forth in SEQ ID NO:13, that is capable of specifically binding to at least a portion of an IAP with a candidate inhibitor or candidate enhancer and detecting caspase enzymatic activity, wherein a decrease in enzymatic activity as compared to a control indicates the presence of an inhibitor and an increase in enzymatic activity indicates the presence of an enhancer. In certain embodiments, the caspase enzymatic activity detected is caspase-3, caspase-7, or caspase-9. In some aspects, the caspase enzymatic activity detected is the presence of a substrate cleavage product produced by a caspase cleavage of a substrate. In specific embodiments, the substrate is acetyl DEVD-aminomethyl coumarin.

Another aspect of the invention provides a method of identifying an inhibitor or enhancer of a caspase-mediated apoptosis comprising contacting a cell containing a vector expressing a polypeptide of the invention with a candidate inhibitor or enhancer and detecting caspase enzymatic activity, wherein a decrease in enzymatic activity as compared to a control indicates the presence of an inhibitor and an increase in enzymatic activity indicates the presence of an enhancer. In certain embodiments, the caspase enzymatic activity detected is caspase-3, caspase-7, or caspase-9. In specific embodiments, the caspase enzymatic activity detected is the presence of a substrate cleavage product produced by a caspase cleavage of a substrate. In certain embodiments, the substrate is acetyl DEVD-aminomethyl coumarin.

In another aspect of the invention, the invention provides a method for identifying a compound that inhibits a peptide or polypeptide containing a consensus IAP-binding motif, as set forth in SEQ ID NO:13, that specifically binds at least a portion of an IAP from binding to said portion of an IAP, comprising contacting a candidate compound with said peptide or polypeptide in the presence of said portion of an IAP and detecting displacement or inhibition of binding of said portion of an IAP from said peptide or polypeptide. In certain aspects, the portion of an IAP is a BIR3 domain while in related aspects, the portion of an IAP is a full length IAP.

In another aspect of the invention, the present invention provides a method for identifying a compound that inhibits a peptide or polypeptide containing a consensus IAP-binding motif, as set forth in SEQ ID NO:13, that specifically binds at least a portion of an IAP from binding to said portion of an IAP, comprising contacting a candidate compound with said peptide or polypeptide in the presence of said portion of an IAP and performing a functional assay that confirms displacement of said portion of an IAP from said peptide or polypeptide. In certain embodiments, the functional assay detects the presence of large and small caspase subunits, and therefrom determines the level of caspase processing activity, wherein a decrease in processing confirms displacement. In specific aspects, the caspase detected is caspase-3, caspase-7, or caspase-9. In some embodiments, the functional assay detects the presence of a substrate cleavage product produced by a caspase cleavage of a substrate. In specific embodiments, the substrate is acetyl DEVD-aminomethyl coumarin.

In yet another aspect of the invention, the invention provides a composition comprising a nucleic acid molecule of the invention and a physiologically acceptable carrier. In a related aspect, the composition contains an expression vector of the invention and a physiologically acceptable carrier.

In another aspect of the invention, the invention provides a composition comprising a peptide of the invention and a physiologically acceptable carrier.

In another aspect of the invention, the present invention provides a composition comprising an antibody of the invention and a physiologically acceptable carrier.

In another aspect, the invention provides a composition comprising an inhibitor or enhancer of apoptosis identified by a method provided by the present invention.

Yet another aspect of the invention provides a method of producing a compound for inhibiting or enhancing apoptosis in a cell, comprising identifying an inhibitor or enhancer of apoptosis according to a method of the invention and purifying the inhibitor or enhancer.

In a related aspect, the invention also provides a process for the manufacture of a compound for inhibiting or enhancing apoptosis in a cell, comprising identifying an inhibitor or enhancer of apoptosis according to a method of the invention, derivitizing the compound, and optionally repeating at least the identification or derivitization steps of the process, to produce a compound that inhibits or enhances apoptosis.

These and other aspects of the present invention will become evident upon reference to the following detailed description and attached drawings. In addition, the various references set forth herein describe more detail certain procedures and compositions (e.g. plasmids, etc.) and are, therefore, incorporated by reference.

### BRIEF DESCRIPTION OF THE DRAWINGS

FIG. 1 is a schematic representation of the human procaspase-9 (SEQ ID NO:18) and the positions of the autocatalytic cleavage site (Asp315, arrow) and caspase-3 cleavage site (Asp330, star) within the linker region (LR) between the large and small subunits. The position of the minor autocatalytic cleavage site (Glu306, circle) is also shown.

FIG. 2 is a scanned image of a Coomassie stained gel representing recombinant WT and mutant caspase-9 variants (lanes 2-4) purified on Talon-affinity resin from bacterial extracts. Lane 1 shows a molecular mass marker; lane 2 illustrates wild type caspase-9; lane 3 illustrates the triple mutant procaspase-9 (E306/D315/D330A) in which Glu306, Asp315, and Asp330 were mutated to Ala, and lane 4 depicts the control unprocessed active site mutant, C287A caspase-9.

FIG. 3 is a scanned image of an autoradiogram representing western blot analysis of the processing of procaspase-3 by recombinant WT and triple mutant caspase-9 proteins in the presence of cytochrome and dATP and in the presence (+) or absence (−) of recombinant Apaf-1.

FIG. 4 is a line graph representation of the activation of the DEVD-AMC cleaving caspases in caspase-9-depleted S100 extracts by the WT and triple mutant caspase-9 in the presence of Apaf-1, in the presence (WT, Triple Mut) or absence (Controls) of cytochrome c and dATP.

FIG. 5 is a scanned image of an autoradiogram representing SDS-PAGE analysis of ³⁵S-labeled procaspase-3 C163A processing by fully processed WT and the uncleavable triple mutant (E306/D315/330A) caspase-9 proteins in the presence of increasing amounts of XIAP.

FIG. 6 is scanned images of western blot analysis using Apaf-1, caspase-9, or XIAP antibodies of gel-filtration analysis of the Apaf-1-caspase-9 holoenzyme complex formed with WT or uncleavable caspase-9, in the presence (panels I and II) or absence (panel III) of XIAP.

FIG. 7 is a scanned image of ³⁵S-labeled procaspase-3 C163A processing by caspase-9-Apaf-1 holoenzyme complexes containing WT caspase-9 in the presence (I) or absence (II) of XIAP or the triple mutant caspase-9 in the presence of XIAP (III).

FIG. 8 is scanned images of immunoblot analysis using an XIAP (upper panel) or caspase-9 (lower panel) antibody of XIAP binding to the Apaf-1-caspase-9 holoenzyme complex containing either WT or uncleavable caspase-9, following immunoprecipitation with an anti-Apaf-1 antibody.

FIG. 9 is a colinear alignment of the N-terminal sequences of Drosophila Reaper (SEQ ID NO:2), Grim (SEQ ID NO:3) and Hid (SEQ ID NO:4), mouse caspase-9-p12 (SEQ ID NO:5), human caspase-9-p12 (SEQ ID NO:6), xenopus caspase-9-p12 (SEQ ID NO:7) and human Smac/DIABLO (SEQ ID NO:8). The BIR3 binding motif is highlighted.

FIG. 10 is a scanned image of an autoradiogram representing SDS-PAGE analysis of the interaction of ³⁵S-labeled XIAP or its isolated BIR3 domain with GST-tagged caspase-9-p12 (residues 316-416), linker region (residues 316-330), p10 (residues 331-416) or mature Smac/DIABLO. The caspase-9 deletion mutants are represented by bar diagrams above the panel.

FIG. 11 is scanned image of an autoradiogram illustrating Far western blot analysis of ³⁵S-labeled XIAP binding to WT and mutant caspase-9 variants or Smac/DIABLO GST fusion proteins immobilized on a nitrocellulose membrane. The GST fusions include WT, triple mutant (E306/D315/D330A), double mutant (D315/D330A), or single mutant (D315A) caspase-9, p12, p10, or full length Smac/DIABLO. The lower panel is a scanned image of a Coomassie stained gel of all the indicated proteins. The arrow indicates p12 of caspase-9. The asterisk indicates p14 of caspase-9, which is generated by processing at E306.

FIGS. 12A and 12B are scanned images representing SDS-PAGE analysis of the interaction of recombinant WT caspase-9, caspase-9 AT316, 317SG or AT316, 317GG mutants or caspase-9 Δlinker mutant with XIAP. FIG. 12A is a scanned image of a Coomassie stained gel of all the indicated recombinant proteins. FIG. 12B is a scanned image of Far western analysis of the indicated recombinant proteins using ³⁵S-labeled XIAP.

FIG. 13 is a bar graph representation of enzymatic activity of recombinant WT caspase-9, caspase-9 AT316, 317SG or AT316, 317GG mutants, caspase-9 Δlinker mutant or caspase-9 triple mutant, in the presence (+) or absence (−) of XIAP-BIR3. The data are represented in % activity relative to the DEVD-AMC cleaving activity in the absence of BIR3.

FIGS. 14A and 14B are scanned images representing SDS-PAGE analysis of the interaction of WT caspase-3 or caspase-3 SG176, 177AV or SGVD176-179AVPF mutant proteins with XIAP. FIG. 14A is a scanned image of a Coomassie stained gel of all the indicated recombinant proteins. FIG. 14B is a scanned image of Far western analysis of the indicated recombinant proteins using ³⁵S-labeled XIAP.

FIG. 15 is a numeric representation of the effect of purified BIR3 or BIR1-BIR2 proteins on enzymatic activity of WT caspase-3 or caspase-3 SG176, 177AV or SGVD176-179AVPF mutant proteins. The IC₅₀s were calculated from the percentage of inhibition.

FIG. 16 is a scanned image depicting the inhibition of BIR3 interaction with Smac/DIABLO and p12 by the linker and Smac-N7 peptides. Left panel, Smac-GST was immobilized onto glutathione resin and then incubated with BIR3 in the absence of any peptide (lane 1, buffer), or presence of linker peptide (lane 2, linker, ATPFQEGLRTFDQLD (SEQ ID NO:11) or non-specific peptide (lane 3, control, MKSDFYFQK (SEQ ID NO:14). Right panel, p12-GST was immobilized onto glutathione resin and then incubated with BIR3 in the absence of any peptide (lane 1, buffer), or presence of Smac-N7 peptide (lane 2, Smac-N7, AVPIAQK (SEQ ID NO:12) or non-specific peptide (lane 3, control). The interactions were analyzed as in FIG. 10.

FIG. 17 is a scanned image of the interaction of ³⁵S-labeled WT or E314S mutant BIR3 domains of XIAP with caspase-9-p12 and mature Smac/DIABLO GST fusion proteins.

FIG. 18 is a bar graphic representation of the effect of the linker peptide (SEQ ID NO:), p12-N5 peptide (ATPFQ (SEQ ID NO:19) and Smac-N5 (AVPIA (SEQ ID NO:20) on cytochrome c-mediated caspase-3 activation in the presence of XIAP.

FIGS. 19A and 19B are proposed models of caspase-9 binding and inhibition by XIAP. FIG. 19A illustrates the conserved binding of BIR3 by caspase-9 and by Smac/DIABLO. The N-terminal tetra-peptides from Smac/DIABLO (AVPI, SEQ ID NO:21) and the p12 subunit of the human caspase-9 (ATPF, SEQ ID NO:28) are shown. Two critical residues on the BIR3 domain, W310 and E314, are highlighted. On the basis of the crystal structure of a Smac-BIR3 complex, the N-terminal tetra-peptide of Smac/DIABLO was replaced by that from the p12 subunit of human caspase-9. Limited energy minimization was performed on the complex between BIR3 and the tetra-peptide from the p12 subunit of the human caspase-9. The N-terminal tetra-peptide (AVPY, SEQ ID NO:15) from the p12 subunit of the rat or mouse caspase-9 more closely resembles the Smac/DIABLO peptide (AVPI, SEQ ID NO:21). FIG. 19B shows the proposed model of caspase-9 inhibition by XIAP. The dimer of mature caspase-9 (based on the atomic coordinates of caspase-3, PDB code IDD1) is represented and the BIR3 domain of XIAP are represented. The approximate location of the catalytic residue on caspase-9, C287, is highlighted. The catalytic site is identified with a circle. H343, which is implicated in binding the catalytic site of capase-9, is also shown.

FIGS. 20A and 20B are bar graphic representations of the effect of IAP-binding peptides and Smac/DIABLO on XIAP-BIR3 inhibition of the caspase-3-AVPF mutant, as measured by cleavage of the peptide substrate DEVD-AMC. The caspase activity in all samples is plotted as a percentage of the activity of caspase-3 in the absence of XIAP-BIR3 (100%).

### DETAILED DESCRIPTION OF THE INVENTION

Prior to setting forth the invention, it may be helpful to an understanding thereof to set forth definitions of certain terms that will be used hereinafter.

An “isolated nucleic acid molecule” refers to a polynucleotide molecule in the form of a separate fragment or as a component of a larger nucleic acid construct, which has been separated from its source cell (including the chromosome it normally resides in) at least once, and preferably in a substantially pure form. Nucleic acid molecules may be comprised of a wide variety of nucleotides, including DNA, RNA, nucleotide analogues, or a combination thereof.

As used herein, a “peptide” is an amino acid sequence of between two and ten contiguous amino acids, including all integer values in between, e.g., 2, 4, 5, 6, 7, 8, 9 and 10 contiguous amino acids. A “polypeptide” is an amino acid sequence of more than ten contiguous amino acids, e.g., 11, 15, 20, 30, 40, 60, 75, 100, 125, 150, 160, 175, 190, 200 or more contiguous amino acids.

A “functional equivalent” of a peptide or polypeptide is a peptide or polypeptide with at least one amino acid substitution that retains at least one functional activity associated with the native peptide or polypeptide. In some circumstances, the functional activity is the specific binding to at least a portion of an IAP. For example, any peptide or polypeptide containing an N-terminal consensus sequence set forth in SEQ ID NO:13 is a functional equivalent and can substitute for any other peptide or polypeptide containing an N-terminal consensus sequence set forth in SEQ ID NO:13. In certain other circumstances, the functional activity is serine protease activity.

A “caspase” refers to a cysteine protease that specifically cleaves proteins after Asp residues. Caspases are initially expressed as zymogens, in which a large subunit is N-terminal to a small subunit. Caspases are generally activated by cleavage at internal Asp residues. These proteins have been identified in many eukaryotes, including C. elegans, Drosophila, mouse, and humans. Currently, there are at least 14 known caspase genes, named caspase-1 through caspase-14. Caspases are found in myriad organisms, including human, mouse, insect (e.g., Drosophila), and other invertebrates (e.g., C. elegans). In Table 1, ten human caspases are listed along with their alternative names.

| Caspase | Alternative name |
|---|---|
| Caspase-1 | ICE |
| Caspase-2 | ICH-1 |
| Caspase-3 | CPP32, Yama, apopain |
| Caspase-4 | ICEᵣₑₗII; TX, ICH-2 |
| Caspase-5 | ICEᵣₑₗIII; TY |
| Caspase-6 | Mch2 |
| Caspase-7 | Mch3, ICE-LAP3, CMH-1 |
| Caspase-8 | FLICE; MACH; Mch5 |
| Caspase-9 | ICE-LAP6; Mch6 |
| Caspase-10 | Mch4, FLICE-2 |

References to procaspase-9 and caspase-9, herein, are intended to include peptides of any origin which are substantially homologous to and which are biologically or functionally equivalent to the procaspase-9 and caspase-9 peptides and polypeptides characterized and described herein. Caspase-9 includes unprocessed procaspase-9, as well as processed caspase-9 subunits, i.e. p35, p12, and p10. In addition, caspase-9 peptides and polypeptides include caspase-9 mutants, fragments, and variants. A peptide “substantially homologous” to another peptide preferably has at least 70-99% amino acid identity, including all integer values in between, e.g., at least 70%, 75%, 80%, 90%, 92%, 95%, 97%, 98% or 99% amino acid identity, with the other peptide. Percent identity is determined utilizing default parameters. Amino acid sequence identity may be determined by standard methodologies, including those set forth supra as well as the use of the National Center for Biotechnology Information BLAST 2.0 search methodology (Altschul et al., J. Mol. Biol. 215:403-10, 1990). In one embodiment, BLAST 2.0 is utilized with default parameters. A preferred method of sequence alignment uses the GCG PileUp program (Genetics Computer Group, Madison, Wis.) (Gapweight: 4, Gaplength weight: 1). The pileUp program creates a multiple sequence alignment from a group of related sequences using progressive, pairwise alignments. PileUp creates a multiple sequence alignment using the progressive alignment method of Feng and Doolittle (J. Mol. Evol. 25:351-360, 1987) and is similar to the method described by Higgins and Sharp (CABIOS 5:151-153, 1989). Further, whether an amino acid change results in a functional peptide can be readily determined by assaying biological properties of the disclosed peptides. For example, the biological properties of caspase-9 functional equivalents can be assayed by determining whether they bind to at least a portion of a IAP, as described in Example 2-4, for example.

A molecule is said to “specifically bind” to a particular peptide or polypeptide if it binds at a detectable level with the particular peptide polypeptide, but does not bind detectably with another polypeptide containing an unrelated sequence. An “unrelated sequence,” as used herein, refers to a sequence that is at most 10% identical to a reference sequence.

The term “in vitro” refers to cell free systems.

The term “derivitizing” or “derivatizing” refers to standard types of chemical modifications of a compound to produce another structurally related compound typically carried out in the process of compound optimization. The resulting structurally related compound is referred to as a “derivative compound.” The current invention includes compositions comprising nucleic acids encoding and peptides and polypeptides corresponding to a peptide of caspase-9, or variants thereof, that retains at least one functional activity associated with caspase-9. All nucleic acids, peptides, and polypeptides of the invention may comprise, consist essentially of, or consist of their defining polynucleotides and/or amino acid sequences. In one embodiment, a peptide or polypeptide has at least two contiguous amino acid residues derived from residues 316-317 or 331-332 of SEQ ID NO:1. The invention also includes antibodies directed to peptides and polypeptides of the invention, as well as compositions comprising nucleic acids, peptides, polypeptides, and antibodies of the invention. In addition, the invention provides methods of using compositions of the invention to modulate apoptosis, to identify modulators of apoptosis, and in therapeutic uses.

A. Caspase-9 Peptides and Polypeptides

The present invention provides a variety of peptides and polypeptides of caspase-9, and variants thereof. Peptides and polypeptides of the invention generally possess one or two specific functional activities associated with caspase-9: (1) the ability to bind to at least a portion of an Inhibitor of Apoptosis Protein (IAP); or (2) cysteine protease activity. In certain embodiments, peptides and polypeptides of the invention include a functional domain or fragment of a caspase-9 that retains the ability to bind at least a portion of an IAP or cysteine protease activity. In other embodiments, peptides and polypeptides of the invention include mutants of wild type caspase-9, in which one of these two wild type functional activities is diminished or completely lacking. The invention also provides other peptides and polypeptides that share at least one of these functional activities with caspase-9. Thus, certain other peptides and polypeptides of the invention are capable of binding to at least a portion of an IAP, while others possess cysteine protease activity. Furthermore, the invention provides fusion proteins that possess both the ability to bind at least a portion of an IAP and cysteine protease activity.

Certain peptides and polypeptides of the invention specifically bind to at least a portion of an IAP. This portion of an IAP is preferably a BIR domain, and it may be BIR1, BIR2, BIR3, or any combination thereof. In addition, preferred peptides and polypeptides of the invention are also capable of binding to a full length IAP. The ability to bind to at least a portion of an IAP may be predicted based upon the amino acid sequence of a peptide or polypeptide, and it may be determined experimentally. Comparison of the amino acid sequence of several polypeptides that are capable of binding to at least a portion of an IAP has revealed one consensus IAP binding domain. These polypeptides include the N-terninal sequences of the Drosophila proteins Reaper (SEQ ID NO:2), Grim (SEQ ID NO:3), and Hid (SEQ ID NO:4), mouse caspase-9-p12 (SEQ ID NO:5, xenopus caspase-9-p12 (SEQ ID NO:7), human Smac/DIABLO (SEQ ID NO:8), human Omi (SEQ ID NO:9), and human Veto (SEQ ID NO:10). The consensus sequence resulting from a colinear alignment of these sequences is the tetra-peptide Ala—Xaa₁—Xaa₂—Xaa₃, wherein Xaal is Val, Thr, or Ile, —Xaa₂ is Pro or Ala, and Xaa₃ is a non-polar or uncharged polar amino acid residue, as set forth in SEQ ID NO:13. The ability of a peptide or polypeptide to bind to at least a portion of an IAP can be determined experimentally by a variety of methods well known in the art. These methods include, for example, in vitro binding assays such as pull-down assays using radio-labeled in vitro translated polypeptides and glutathione-S-transferase (GST)-BIR fusion proteins and co-immunoprecipitation assays using epitope-tagged polypeptides. Detailed descriptions of preferred methods of examining the capability of a peptide or polypeptide to bind to at least a portion of an IAP are provided in Examples 1-4 and 6. Such methods are also described in Srinivasula, S. M. et al. J Biol Chem 275:36152-36157, 2000, which is hereby incorporated by reference.

Certain peptides and polypeptides of the invention possess cysteine protease activity. Preferably, these peptides and polypeptides possess a cysteine protease functional domain of a caspase. Certain peptides and polypeptides of the invention are fusion proteins wherein an IAP binding domain is fused to a polypeptide possessing cysteine protease activity. In other embodiments, these peptides and polypeptides may be mutants of wild type polypeptides, wherein the mutation diminishes or abolishes IAP binding. Where a peptide or polypeptide of the invention possesses both cysteine protease activity and the capability to bind to at least a portion of an IAP, binding to at least a portion of an IAP preferably inhibits said cysteine protease activity. Cysteine protease activity may be predicted by the presence of the consensus cysteine protease active site pentapeptide, Gln-Ala-Cys-Xaa-Gly, wherein Xaa is Arg, Gln, or Gly (SEQ ID NO:17). In addition, cysteine protease activity may be experimentally determined by a variety of methods well known in the art. Such methods include, for example, enzymatic assays measuring the ability of a bacterially expressed polypeptide to cleave an appropriate substrate, such as DEVD-aminomethyl coumarin. Detailed descriptions of preferred methods of determining cysteine protease activity are provide in Examples 1 and 2, as well as in Srinivasula, S. M. et al.

The present invention includes caspase-9 peptides and polypeptides that are capable of binding to at least a portion of an IAP. Such polypeptides may be used for a variety of purposes, such as, for example, to inhibit IAP binding to and inhibition of wild-type caspase-9. These polypeptides may, therefore, be used to promote apoptosis, in certain situations. In most circumstances, such peptides and polypeptides lack cysteine protease activity. The invention does not include full length wild-type procaspase-9. Preferred peptides and polypeptides comprise at least the N-terminal four amino acid residues of the caspase-9-p12 subunit, as set forth in amino acid residues 316 through 319 of SEQ ID NO:1, or at least the N-terminal two to four amino acid residues of the caspase-9-plO subunit, as set forth in amino acid residues 331 through 332 or 331 through 334 of SEQ ID NO:1. In addition, the invention includes polypeptides comprising the caspase-9 linker region, as set forth in SEQ ID NO:11. Caspase-9 peptides and polypeptides of the invention that are capable of binding to at least a portion of an IAP may further comprise additional caspase-9 amino acid sequence. Preferably, the caspase-9 peptides and polypeptides lack at least one wild type caspase-9 functional activity. Preferably, this is cysteine protease activity. In certain embodiments, caspase-9 polypeptides of the invention comprise at least the N-terminal four amino acid residues of the caspase-9-p12 subunit, as set forth in amino acid residues 316 through 319 of SEQ ID NO:1, as well as up to an additional 97 contiguous C-terminal amino acid residues derived from resides 320 through 416 of SEQ ID NO:1, including all integer values in between, e.g., 2, 4, 5, 7, 9, 10, 15, 20, 30, 40, 50, 60, 70, 80, or 96. In other embodiments, polypeptides of the invention comprise at least the N-terminal two to four amino acid residues of the caspase-9-p10 subunit, as set forth in amino acid residues 331 through 332 or 331 through 334 of SEQ ID NO:1, as well as up to an additional 82 through 84 contiguous C-terminal amino acid residues derived from residues 333 through 416 of SEQ ID NO:1, including all integer values in between, e.g., 2, 4, 5, 7, 9, 10, 15, 20, 30, 40, 50, 60, 70, 80, 81, 82, or 83. Caspase-9 polypeptides of the invention include caspase-9-p12 and caspase-9-p10 subunits. Caspase-9 polypeptides of the invention comprising any of amino acid residues 316 through 416 of SEQ ID NO:1, that are capable of binding to at least a portion of an IAP, may also include additional N-terminal caspase-9 amino acid residues. However, caspase-9 polypeptides of the invention containing such additional N-terminal sequence preferably lack cysteine protease activity. Thus, the additional N-terminal amino acid residues preferably are lacking specific amino acid sequences required for cysteine protease activity. Such sequences may be lacking due to amino acid insertions, deletions, or substitutions, for example. One preferred mutation contains a substitution of the active site Glu306 of SEQ ID NO:1 with an Ala.

The invention also includes mutant procaspase-9 polypeptides that lack the ability to bind to at least a portion of an IAP. Such polypeptides may contain mutations that inhibit their normal proteolytic processing. Absent proteolytic processing to reveal the IAP binding sites located at the N-terminus of the p12 and p10 subunits, such procaspase-9 mutants typically are unable to bind to or be inhibited by an IAP. In addition, these polypeptides preferably possess cysteine protease activity. Such mutants may be used for a variety of purposes, including deregulated caspase-9 polypeptides. Because such polypeptides are not subject to inhibition by an IAP, they may be used to promote apoptosis in certain situations. A variety of procaspase-9 mutants that fail to undergo normal processing are included within the invention. Sequence analysis of purified recombinant caspase-9 revealed that >90% of caspase-9 processing in bacteria occurs at Asp315 of SEQ ID NO:1, which generates the p35 and p12 subunits, and the remaining 10% of processing occurs at Asp330 to generate the plO subunit. A minor processing was also detected at Glu306. The invention includes procaspase-9 mutants that fail to undergo normal processing at one or more proteolytic sites. Thus, preferred procaspase-9 mutants that fail to undergo normal processing include a triple mutant procaspase-9 containing amino acid substitutions of the amino acid residues Asp315, Asp330, and Glu306, as set forth in SEQ ID NO:1. Each of these amino acid residues may also be mutated individually to generate single mutants, and two of theses residues may be mutated to generate double mutants. A preferred single mutant contains amino acid residue 315 of SEQ ID NO:1 substituted by another amino acid residue, while a preferred double mutant contains amino acid residues 315 and 330 of SEQ ID NO:1 substituted by other amino acid residues. In certain embodiments of the invention, procaspase-9 processing mutants have the amino acid residue Ala substituted for one or more of residues Asp315, Asp330, and Glu 306, as set forth in SEQ ID NO:1. Other procaspase-9 mutants that fail to undergo normal processing include deletion mutants lacking one or more proteolytic cleavage sites. Deletions may include one or more proteolytic sites, and they may be as small as one amino acid residue or larger. One preferred procaspase-9 deletion mutant lacks the linker region (amino acid residues 316 through 330 of SEQ ID NO:1). One of ordinary skill in the art would recognize that there are a wide variety of mutants could be generated that lacked normal processing, including mutants with amino acid substitutions, deletions, and/or insertions. Preferably, peptides corresponding to procaspase-9 mutants that fail to undergo normal processing, or variants thereof, lack the ability to bind an IAP or a portion of an IAP. However, such procaspase-9 mutants may include the cysteine protease active site of caspase-9 and may possess cysteine protease catalytic activity. Mutant procaspase-9 polypeptides that fail to bind to at least a portion of an IAP include both mutants of a full length caspase-9 and mutants of less than full length fragments of a caspase-9.

In addition, fusion proteins containing an N-terminal region of a caspase-9-p12 or caspase-9-p10 subunit, or a variant thereof, are included within the invention, wherein the fusion protein is capable of specifically binding to at least a portion of an IAP. Fusion proteins may contain a variety of different polypeptides fused to a p12 or p10 sequence, including for example, at least a portion of a caspase. One preferred fusion protein includes a portion of a procaspase-9 that specifically binds at least a portion of an IAP, as well as a portion of a procaspase-9 that contains a mutated cysteine protease active site, such that the expressed fusion protein is capable of binding at least a portion of an IAP but lacks cysteine protease activity. Preferred caspase-3 fusion proteins exhibit caspase-3 enzymatic activity that is capable of being at least partially inhibited by an IAP or an IAP BIR3 domain. Such caspase-3 fusion proteins preferably contain a p12 N-terminal amino acid sequence of Ala-Val or any four residues set forth in SEQ ID NO:13. This sequence may be in addition to or in substitution for the same number of wild type caspase-3 p12 N-terminal amino acid residues.

A variety of polypeptide sequences capable of binding to at least a portion of an IAP are provided by the invention. These include the N-terminal sequences of the Drosophila proteins Reaper (SEQ ID NO:2), Grim (SEQ ID NO:3), and Hid (SEQ ID NO:4), mouse caspase-9-p12 (SEQ ID NO:5, xenopus caspase-9-p12 (SEQ ID NO:7), human Smac/DIABLO (SEQ ID NO:8), human Omi (SEQ ID NO:9), and human Veto (SEQ ID NO:10). The consensus sequence resulting from a colinear alignment of these sequences is set forth in SEQ ID NO:13 as Ala—Xaa₁—Xaa₂-Xaa₃, wherein Xaal is Val, Thr, or Ile, Xaa₂ is Pro or Ala, and Xaa₃ is a non-polar or uncharged polar amino acid residue. Peptides and polypeptides of the invention may comprise each of these specific identified N-terminal amino acid sequences with similarity to the N-terminus of human caspase-9-p12, as well as all other amino acid sequences with the identified consensus sequence that are also capable of binding to at least a portion of an IAP. Polypeptides of the invention generally contain a tetrapeptide set forth in SEQ ID NO:13 at their N-terminus. However, the tetrapeptide may be located internally or at the C-terminus provided the resulting peptide or polypeptide is capable of binding to at least a portion of an IAP. Further, peptides and polypeptides of the invention that contain an IAP-binding tetrapeptide motif may contain additional contiguous or non-contiguous amino acid sequences corresponding to any of the native proteins identified above, which contain such a binding motif. Preferably, though, these will not correspond to full length wild type proteins. A preferred nucleic acid molecule of the invention encodes a peptide or polypeptide corresponding to the seven N-terminal amino acid residues of Smac/DIABLO, as set forth in SEQ ID NO:12.

The current invention encompasses all variants (including alleles) of the caspase-9 peptides and polypeptides of the invention. Preferably, such variants are functional variants that retain at least one biological or functional activity associated with caspase-9. Preferably, the retained biological or functional activity is either the ability to bind to at least a portion of an Inhibitor of Apoptosis Protein (IAP) or cysteine protease activity. Such functional variants may result from natural polymorphisms or may be synthesized, preferably by recombinant methodology, and differ from wild-type peptides or polypeptides by one or more amino acid substitutions, insertions, deletions, or the like. Amino acid changes in functional variants of caspase-9 peptides or polypeptides may be conservative substitutions. Guidance in determining which amino acid residues can be substituted, inserted, or deleted without abolishing biological or immunological activity can be found using computer programs well known in the art, such as DNASTAR software. Preferably, amino acid changes in functional variants are conservative amino acid changes, i.e., substitutions of similarly charged or uncharged amino acids. It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant. Whether an amino acid change results in a functional protein or polypeptide can readily be determined by testing the altered protein or polypeptide in a biological assay, such as, for example, an in vitro binding assay or a cysteine protease enzymatic assay, as described herein. Variants can also include post-translational modifications. Caspase-9 variants include variants of all caspase-9 peptides and polypeptides, including fragments and functional domains of caspase-9. Variants of a caspase-9 peptide or polypeptide include peptides and polypeptides containing a consensus IAP-binding motif, as set forth in SEQ ID NO:13, wherein the peptide or polypeptide is capable of binding to at least a portion of an IAP.

Conservative amino acid changes involve the substitution of one amino acid for another amino acid of a family of amino acids with structurally related side chains. Naturally occurring amino acids are generally divided into four families: acidic (aspartate, glutamate); basic (lysine, arginine, histidine); non-polar (alanine, valine, leucine, isoleucine, proline, phenylalanine, methionine, tryptophan); and uncharged polar (glycine, asparagine, glutamine, cysteine, serine, threonine, tyrosine) amino acids. Phenylalanine, tryptophan, and tyrosine are sometimes classified as aromatic amino acids. Non-naturally occurring amino acids can also be used to form protein variants of the invention.

In the region of homology to the native sequence, functional variants preferably have at least 70-99% amino acid identity, including all integer values in between, e.g., at least 70%, 75%, 80%, 90%, 92%, 95%, 97%, 98%, or 99% amino acid identity. In certain embodiments, the peptide or polypeptide sequence is compared to a test sequence, or, when necessary, a particular domain is compared to a test sequence to determine percent identity, typically by utilizing default parameters. Amino acid sequence identity may be determined by standard methodologies, including those set forth supra as well as the National Center for Biotechnology Information BLAST 2.0 search methodology (Altschul et al., J. Mol. Biol. 215:403-10, 1990). In one embodiment, BLAST 2.0 is utilized with default parameters. A preferred method of sequence alignment uses the GCG PileUp program (Genetics Computer Group, Madison, Wisconsin) (Gapweight: 4, Gaplength weight: 1). The pileUp program creates a multiple sequence alignment from a group of related sequences using progressive, pairwise alignments. PileUp creates a multiple sequence alignment using the progressive alignment method of Feng and Doolittle (J. Mol. Evol. 25:351-360, 1987) and is similar to the method described by Higgins and Sharp (CABIOS 5:151-153, 1989).

Caspase-9 functional variants can include hybrid and modified forms of caspase-9 peptides or polypeptides such as, but not limited to, fusion polypeptides. Caspase-9 fusion polypeptides include peptides or polypeptides of caspase-9 fused to amino acid sequences comprising one or more heterologous polypeptides. Such heterologous polypeptides may correspond to naturally occurring polypeptides of any source or may be recombinantly engineered amino acid sequences. Fusion proteins are useful for purification, generating antibodies against amino acid sequences, and for use in various assay systems. For example, fusion proteins can be used to identify proteins or a domain of that protein which interacts with a peptide or polypeptide of the invention or which interferes with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain can be used to target other protein domains to cellular locations in which the domains are not normally found, such as bound to a cellular membrane or secreted extracellularly.

A fusion protein comprises two or more peptide or polypeptide segments fused together by means of a peptide bond. A first amino acid sequence for use in fusion proteins of the invention can be selected from any contiguous amino acid sequence described herein. The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex A DNA binding domain (DBD) fusions, GAL4 DNA binding domain fusions, and herpes simplex virus (HSV) BP16 protein fusions.

Fusion proteins of the invention can be made, for example, by covalently linking two protein segments or by standard procedures in the art of molecular biology. Recombinant DNA methods can be used to prepare fusion proteins, for example, by making a DNA construct comprising nucleotides encoding a first polypeptide fused in-frame to nucleotides encoding a second polypeptide and expressing the DNA construct in a host cell, as is well known in the art. Vectors and kits for constructing fusion proteins are commercially available from a variety of sources, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Caspase-9 peptides and polypeptides of the invention may be fused to a wide variety of heterologous peptides or polypeptides, not limited to those described above. Heterologous peptides and polypeptides may be of any length and may include one or more amino acids. In certain embodiments, caspase-9 fusion proteins may be produced to facilitate expression or purification. For example, a caspase-9 polypeptide may be fused to maltose binding protein or glutathione-S-transferase. In other embodiments, caspase-9 fusion proteins may contain an epitope tag to facilitate identification or purification. One example of a tag is the FLAG epitope tag (Kodak).

Peptides and polypeptides of the invention may be produced by any means available in the art and are typically produced using recombinant DNA protein expression methodologies widely known and available in the art. Synthetic chemistry methods, such as solid phase peptide synthesis can also be used to synthesize proteins, fusion proteins, or polypeptides of the invention.

Recombinantly expressed peptides and polypeptides of the invention can be purified from culture medium or from extracts of cultured cells. Methods of protein purification such as affinity chromatography, ionic exchange chromatography, HPLC, size exclusion chromatography, ammonium sulfate crystallization, electrofocusing, or preparative gel electrophoresis are well known and widely used in the art (see generally Ausubel et al., supra; Sambrook et al., supra). An isolated purified protein is generally evidenced as a single band on an SDS-PAGE gel stained with Coomassie blue.

B. Caspase-9 Nucleic Acid Molecules

The present invention provides nucleic acid molecules comprising, consisting essentially of, or consisting of polynucleotides that encode peptides and polypeptides that share one or more functional characteristics with caspase-9. The invention provides nucleic acid molecules corresponding to an isolated polynucleotide fragment encoding a peptide or polypeptide of the invention. In addition, the invention provides cloning vectors and expression vectors containing polynucleotides encoding peptides and polypeptides of the invention. The invention also provides a variety of other nucleic acid molecules, such as isolated antisense RNA molecules and antisense and ribozyme expression vectors, each containing nucleotide sequence corresponding to a peptide or polypeptide of the invention. Nucleic acid molecules of the invention include all types of nucleic acids, including, for example, dsDNA, ssDNA, RNA, and cDNA. Thus, it is understood that the invention includes all nucleic acid molecules encoding any peptide or polypeptide of the invention, or related antisense RNA. Furthermore, all nucleic acid molecules of the invention may comprise, consist essentially of, or consist of the described polynucleotides. Similarly, all polynucleotides of the invention may comprise, consist essentially of, or comprise the described peptides or polypeptides.

Nucleic acids of the invention include all nucleic acid molecules comprising polynucleotides encoding peptides or polypeptides with regions of sequence identical or similar to the N-terminus of the human caspase-9-p12 subunit, as set forth in amino acid residues 316 through 416 of SEQ ID NO:1, that are capable of specifically binding to at least a portion of an IAP. In addition, nucleic acid molecules of the invention include polynucleotides encoding at least an N-terminal region corresponding to the N-terminus of the human caspase-9-p10 subunit, as set forth in amino acid residues 331 through 416 of SEQ ID NO:1, and variants thereof, that are capable of binding to at least a portion of an IAP. Polypeptides containing N-terminal regions of caspase-9-p12 or caspase-9-p10 include a variety of molecules capable of binding at least a portion of an IAP, including for example, a caspase-9 linker peptide, as set forth in SEQ ID NO:11. Preferably, polypeptides of the invention containing caspase-9 sequences and capable of binding to a portion of an IAP do not possess wild type caspase-9 serine protease activity. Thus, polynucleotides encoding full length procaspase-9 do not fall within the scope of the invention.

The nucleic acid sequence for full-length human procaspase-9 and the encoded protein sequence are available in GenBank/EBI DataBank at Accession No. XM_{—}048848. The nucleotide sequence encoding human procaspase-9 has been incorporated into the application in SEQ ID NO:16, and the amino acid sequence of human procaspase-9 has been incorporated into the application in SEQ ID NO:1.

Caspase-9 and other nucleic acid molecules of the invention may be isolated from genomic DNA or cDNA according to practices known in the art (see Sambrook and Russell, Molecular Cloning: A Laboratory Manual, Cold Spring Harbor Press, 2001). Nucleic acid probes corresponding to a region of the caspase-9 sequences disclosed in the invention may be used to screen either genomic or cDNA libraries. An oligonucleotide probe suitable for screening genomic or cDNA libraries is generally 20-40 bases in length. The oligonucleotide may be synthesized or purchased commercially. The probe may be labeled with a variety of molecules that facilitate detection, such as a radionuclide (e.g., ³²P), an enzymatic label, a protein label, a fluorescent label, or biotin.

Genomic and cDNA libraries may be constructed in a variety of suitable vectors including, for example, plasmid, bacteriophage, yeast artificial chromosome and cosmid vectors. Alternatively, libraries may be purchased from a commercial source (e.g., Clontech, Palo Alto, Calif.). Libraries may contain genomic DNA or cDNA inserts isolated from any species. Nucleotide probes corresponding to the caspase-9 sequences disclosed in the current application can be used to screen libraries constructed from DNA isolated from other species and, therefore, identify and isolate other caspase-9 nucleic acid molecules within the scope of the current invention.

Other methods of obtaining caspase-9 and other polynucleotide sequences of the invention include polymerase chain reaction (PCR) and expression cloning. One preferred method is to perform PCR to amplify a target nucleic acid molecule from cDNA or genomic DNA using oligonucleotide primers corresponding to the 5′ and 3′ ends of the target nucleic acid molecule or region thereof. Detailed methods of PCR cloning may be found in Ausubel, et al., Current Protocols in Molecular Biology, Greene Publishing Associates and Wiley-Interscience, NY, 1995, for example. A preferred method of expression cloning is to use a polypeptide probe capable of binding a peptide or polypeptide expressed by the target nucleic acid sequence. The probe may comprise antibodies or binding partners specific for the expressed nucleic acid molecule. Methods of expression cloning are described in Sambrook, et al., Molecular Cloning: A Laboratory Manual, Cold Spring Harbor Press, 1989, Ausubel, et al. Current Protocols in Molecular Biology, Greene Publishing Associates and Wiley-Interscience, NY, 1995; and Blackwood and Eisenman, Methods Enzymology 254:229-240, 1995. Expression cloning is a particularly useful procedure to identify functional homologs of different species. For example, antibody probes suitable for cross-species cloning can include those directed against conserved regions of caspase-9 peptides or polypeptides. Preferably, the antibodies will bind to the denatured caspase-9 polypeptide. Polypeptide probes suitable for expression cloning of a caspase-9 peptide or polypeptide, or variant thereof, of the invention include peptides or polypeptides corresponding to at least a portion of an IAP that is specifically bound by caspase-9. Preferably, the portion includes a BIR3 domain of an IAP.

Polynucleotides of the invention may also be made using the techniques of synthetic chemistry given the sequences disclosed herein. The degeneracy of the genetic code permits alternate nucleotide sequences that encode amino acid sequences presented in SEQ ID NO:1. All such nucleotide sequences are within the scope of the present invention.

Isolated genes corresponding to the cDNA sequences disclosed herein are also provided. Methods such as those described above can be used to isolate genes (genomic clones) that correspond to known cDNA sequences. Preferred methods include screening genomic libraries with probes comprising cDNA fragments and PCR amplification of genomic clones from genomic libraries. All polypeptides encoded by the isolated genes are within the scope of the invention. These polypeptides include, but are not limited to, polypeptides encoded by the cDNA set forth in SEQ ID NO:16, isoforms of these polypeptides resulting from alternative splicing of the isolated genes, as well as functional fragments thereof.

Nucleic acid sequences encoding caspase-9 or other peptides or polypeptides of the invention, or variants thereof, may be fused to a variety of heterologous sequences, such as those encoding affinity tags (e.g., GST and His-tag) and those encoding a secretion signal. For instance, when the nucleic acid sequence encoding a caspase-9 peptide or polypeptide is fused to a sequence encoding a secretion signal, the resulting polypeptide is a precursor protein that can be subsequently processed and secreted. The processed caspase-9 peptide or polypeptide may be recovered from the cell lysate, periplasmic space, phloem, or from the growth or fermentation medium. Secretion signals suitable for use are widely available and are well known in the art (e.g., von Heijne, J. Mol. Biol. 184:99-105, 1985).

The nucleic acid molecules of the subject invention also include variants (including alleles) of the native human caspase-9 nucleic acid molecule that is identified in SEQ ID NO:16. Variants of the caspase-9 nucleic acid molecules provided herein include natural variants (e.g., degenerate forms, polymorphisms, splice variants or mutants) and those produced by genetic engineering. Variants generally have at least 75%, 80%, 85%, 90%, 95%, 98% or 99% (including the percentages of all integer value between 70 and 99) nucleotide identity with SEQ ID NO:16. The identity algorithms and settings that may be used are defined herein infra, but percent identity may also be determined using computer programs that employ the Smith-Waterman algorithm, such as the MPSRCH program (Oxford Molecular), using an affine gap search with the following parameters: a gap open penalty of 12 and a gap extension penalty of 1. A preferred method of sequence alignment uses the GCG PileUp program (Genetics Computer Group, Madison, Wis.) (Gapweight: 4, Gaplength weight: 1). In certain embodiments, the alignment algorithm utilizes default parameters. Further, a nucleotide variant will typically be sufficiently similar in sequence to hybridize to the reference sequence under stringent hybridization conditions. For nucleic acid molecules over approximately 50 basepairs, stringent conditions include hybridizing nucleic acid molecules in a solution comprising about 1 M Na⁺ at 25° to 30° C. below the Tm: e.g., 5× SSPE, 0.5% SDS, at 65° C.; and removing insufficiently specific hybridization using the following wash conditions: 2× SSC (0.3 M NaCl, 0.03 M sodium citrate, pH 7.0), 0.1% SDS, room temperature twice, 30 minutes each; then 2× SSC, 0.1% SDS, 50° C. once, 30 minutes; then 2× SSC, room temperature twice, 10 minutes each. Suitable moderately stringent conditions include prewashing in a solution of 5× SSC, 0.5% SDS, 1.0 mM EDTA (pH 8.0); hybridizing at 50° C.-65° C., 5× SSC, overnight; followed by washing twice at 65° C. for 20 minutes with each of 2×, 0.5× and 0.2× SSC containing 0.1% SDS.

Nucleic acid sequences that are substantially the same as the nucleic acid sequences encoding peptides or polypeptides of the invention are included within the scope of the invention. Such substantially same sequences may, for example, be substituted with codons optimized for expression in a given host cell such as E. coli. The invention includes nucleic acid sequences encoding functional domains or fragments of caspase-9 or IAP-binding peptides or polypeptides of the invention. Deletions, insertions and/or nucleotide substitutions within a caspase-9 nucleic acid molecule are also within the scope of the current invention. Such alterations may be introduced by standard methods known in the art such as those described by Ausubel et al., supra. In addition, the invention includes nucleic acids that encode polypeptides that are recognized by antibodies that specifically bind a procaspase-9 or caspase-9 polypeptide or subunit, or fragment thereof.

Exemplary nucleic acids that encode caspase-9 peptides or polypeptides of the present invention have coding sequences set forth in SEQ ID NO:16. Polynucleotide molecules of the invention contain less than a whole chromosome and can be single-stranded or double-stranded. Preferably, the polynucleotide molecules are intron-free. Nucleic acid molecules of the invention can comprise at least 11, 15, 18, 21, 30, 33, 42, 60, 66, 72, 84, 90, 100, 120, 140, 160, 180, 200, 220, 240, 300, 600, 900, 1200, and 1248, and all integer values there between, contiguous nucleotides of the human procaspase-9 gene, the homologues of this gene, the complements of this gene and its homologues, and degenerate forms.

The present invention also includes nucleic acid sequences that will hybridize to sequences that encode human or murine procaspase-9 or complements thereof. The invention includes nucleic acid sequences encoding peptides and polypeptides of at least the N-terminus of the caspase-9-p12 or -p10 subunits, or variants thereof. Deletions, insertions and/or nucleotide substitutions within a procaspase-9 or caspase-9 nucleic acid molecule are also within the scope of the current invention. Such alterations may be introduced by standard methods known in the art such as those described in Ausubel et al., supra. Also included are nucleic acid sequences encoding functional equivalents of a procaspase-9 peptide or polypeptide. In addition, the invention includes nucleic acids that encode polypeptides that are recognized by antibodies that bind a procaspase-9 peptide, polypeptide, functional variants of each, and functional equivalents of each.

Polynucleotide molecules of the invention also include molecules that encode single-chain antibodies that specifically bind to the disclosed peptides, that specifically bind to mRNA encoding the disclosed proteins, and fusion proteins comprising amino acid sequences of the disclosed proteins.

C. Vectors, Host Cells and Means of Expressing and Producing Protein

The present invention encompasses vectors comprising regulatory elements linked to caspase-9 or other polynucleotide sequences of the invention. Such vectors may be used, for example, in the propagation and maintenance of caspase-9 nucleic acid molecules or the expression and production of caspase-9 peptides or polypeptides or functional variants or functional equivalents of each. Vectors may include, but are not limited to, plasmids, episomes, baculovirus, retrovirus, lentivirus, adenovirus, and parvovirus, including adeno-associated virus.

The peptides and polypeptides of the invention, including caspase-9 fragments and mutant caspase-9 polypeptides, may be expressed in a variety of host organisms. In certain embodiments, they are produced in mammalian cells, such as CHO, COS-7, or 293 cells. Other suitable host organisms include bacterial species (e.g., E. coli and Bacillus), other eukaryotes such as yeast (e.g., Saccharomyces cerevisiae), plant cells, baculovirus, and insect cells (e.g., Sf9). Vectors for these hosts are well known in the art.

A DNA sequence encoding a caspase-9 peptide or polypeptide, or a variant or mutant thereof, is introduced into an expression vector appropriate for the host. The desired coding sequence is typically subcloned from an existing clone or synthesized. As described herein, a fragment of the coding region may be used. A preferred means of synthesis is to PCR amplify a nucleic acid molecule encoding the peptide or polypeptide of the present invention from cDNA, genomic DNA, or a recombinant clone, using a set of primers that flank the desired portion of the protein. Restriction sites are typically incorporated into the primer sequences and are chosen with regard to the cloning site of the vector. If necessary, translational initiation and termination codons can be engineered into the primer sequences. The sequence of the coding region can be codon-optimized for expression in a particular host. For example, a caspase-9 cDNA fragment isolated from a human cell that is to be expressed in a fungal host, such as yeast, can be altered in nucleotide sequence to use codons preferred in yeast. Further, it may be beneficial to insert a traditional AUG initiation codon at CUG initiation positions to maximize expression, or to place an optimized translation initiation site upstream of a CUG initiation codon. Such codon-optimization may be accomplished by methods such as splice overlap extension, site-directed mutagenesis, automated synthesis, and the like.

At minimum, an expression vector of the invention must contain a promoter sequence. As used herein, a “promoter” refers to a nucleotide sequence that contains elements that direct the transcription of a linked gene. At minimum, a promoter contains an RNA polymerase binding site. More typically, in eukaryotes, promoter sequences contain binding sites for other transcriptional factors that control the rate and timing of gene expression. Such sites include TATA box, CAAT box, POU box, AP1 binding site, and the like. Promoter regions may also contain enhancer elements. When a promoter is linked to a gene so as to enable transcription of the gene, it is “operatively linked”.

Typical regulatory elements within vectors include a promoter sequence that contains elements that direct transcription of a linked gene and a transcription termination sequence. The promoter may be in the form of a promoter that is naturally associated with the gene of interest. Alternatively, the nucleic acid may be under control of a heterologous promoter not normally associated with the gene. For example, tissue specific promoter/enhancer elements may be used to direct expression of the transferred nucleic acid in repair cells. In certain instances, the promoter elements may drive constitutive or inducible expression of the nucleic acid of interest. Mammalian promoters may be used, as well as viral promoters capable of driving expression in mammalian cells. Examples of other regulatory elements that may be present include secretion signal sequences, origins of replication, selectable markers, recombinase sequences, enhancer elements, nuclear localization sequences (NLS), and matrix association regions (MARS).

The expression vectors used herein include a promoter designed for expression of the proteins in a host cell (e.g., bacterial). Suitable promoters are widely available and are well known in the art. Inducible or constitutive promoters are preferred. Such promoters for expression in bacteria include promoters from the T7 phage and other phages, such as T3, T5, and SP6, and the trp, lpp, and lac operons. Hybrid promoters (see U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. Pat. No. 4,870,009), and the like.

The promoter controlling transcription of caspase-9, or a variant thereof, may itself be controlled by a repressor. In some systems, the promoter can be derepressed by altering the physiological conditions of the cell, for example, by the addition of a molecule that competitively binds the repressor, or by altering the temperature of the growth media. Preferred repressor proteins include, but are not limited to, the E. coli lacI repressor responsive to IPTG induction, the temperature sensitive λcI857 repressor, and the like. The E. coli lacI repressor is preferred.

In other preferred embodiments, the vector also includes a transcription terminator sequence. A “transcription terminator region” has either a sequence that provides a signal that terminates transcription by the polymerase that recognizes the selected promoter and/or a signal sequence for polyadenylation.

Preferably, the vector is capable of replication in the host cells. Thus, when the host cell is a bacterium, the vector preferably contains a bacterial origin of replication. Preferred bacterial origins of replication include the fl-ori and col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host. A selectable marker gene includes any gene that confers a phenotype on the host that allows transformed cells to be identified and selectively grown. Suitable selectable marker genes for bacterial hosts include the ampicillin resistance gene (Amp^{r}), tetracycline resistance gene (Tc^{r}) and the kanamycin resistance gene (Kan^{r}). The kanamycin resistance gene is presently preferred. Suitable markers for eukaryotes usually require a complementary deficiency in the host (e.g., thymidine kinase (tk) in tk-hosts). However, drug markers are also available (e.g., G418 resistance and hygromycin resistance).

The sequence of nucleotides encoding caspase-9, or variants thereof, may also include a secretion signal or the mitochondrial targeting sequence (MTS) sequence can be removed, whereby the resulting peptide or polypeptide is a precursor protein processed and secreted. The resulting processed peptide or polypeptide may be recovered from the periplasmic space, the growth medium, phloem, etc. Secretion signals suitable for use are widely available and are well known in the art (von Heijne, J. Mol. Biol. 184:99-105, 1985). Prokaryotic and eukaryotic secretion signals that are functional in E. coli (or other host) may be employed. The presently preferred secretion signals include, but are not limited to, those encoded by the following E. coli genes: pelB (Lei et al., J. Bacteriol. 169:4379, 1987), phoA, ompA, ompT, ompF, ompC, beta-lactamase, and alkaline phosphatase. Alternatively, a mitochondrial targeting sequence may be recombinantly engineered into an expression vector, such that the expressed protein contains such sequence and is preferentially retained within the mitochondria. Mitochondrial targeting sequences that may be used according to the invention are known in the art and include, for example, those from eukaryotic mitochondrial P450 polypeptides, and the MTS located within the amino terminal 55 amino acids of the Smac/DIABLO precursor polypeptide. Methods of predicting whether a sequence is capable of targeting a polypeptide to the mitochondria are provided in Claros, M. G. and Vincens, P., Computational method to predict mitochondrially imported proteins and their targeting sequence, Eur. J. Biochem. 241, 779-786 (1996).

One skilled in the art appreciates that there are a wide variety of suitable vectors for expression in bacterial cells that are readily obtainable. Vectors such as the pET series (Novagen, Madison, Wis.), the tac and trc series (Pharmacia, Uppsala, Sweden), pTTQ18 (Amersham International plc, England), pACYC 177, the pGEX series, and the like are suitable for expression of Smac. Baculovirus vectors, such as pBlueBac (see, e.g., U.S. Pat. Nos. 5,278,050, 5,244,805, 5,243,041, 5,242,687, 5,266,317, 4,745,051, and 5,169,784; available from Invitrogen, San Diego) may be used for expression in insect cells, such as Spodoptera frugiperda sf9 cells (see U.S. Pat. No. 4,745,051). The choice of a bacterial host for the expression of Smac is dictated in part by the vector. Commercially available vectors are paired with suitable hosts.

A wide variety of suitable vectors for expression in eukaryotic cells are also available. Such vectors include pCMVLacI, pXT1 (Stratagene Cloning Systems, La Jolla, Calif.); pCDNA series, pREP series, pEBVHis (Invitrogen, Carlsbad, Calif.). In certain embodiments, Smac gene is cloned into a gene targeting vector, such as pMClneo, a pOG series vector (Stratagene Cloning Systems).

Caspase-9 and functionally related peptides or polypeptides, as discussed earlier, may be expressed as fusion proteins to aid in purification. Such fusions may be, for example, glutathione-S-transferase fusions, Hex-His fusions, or the like such that the fusion construct may be easily isolated. With regard to Hexa-His fusions, such fusions can be isolated by metal-containing chromatography, such as nickel-coupled beads. Briefly, a sequence encoding His₆ is linked to a DNA sequence encoding Smac. Although the His₆ sequence can be positioned anywhere in the molecule, preferably it is linked at the 3′ end immediately preceding the termination codon. The fusion may be constructed by any of a variety of methods. A convenient method is amplification of the Smac gene using a downstream primer that contains the codons for His₆.

The purified caspase-9 or related peptide or polypeptide may be used in various assays to screen for modulators (i.e., inhibitors or enhancers) of apoptosis. These assays may be performed in vitro or in vivo and utilize any of the methods described herein or that are known in the art. The protein may also be crystallized and subjected to X-ray analysis to determine its 3-dimensional structure. Peptides and polypeptides of the invention described herein may also be used as immunogens for raising antibodies.

Recombinant peptides and polypeptides of the invention, including caspase-9 and related polypeptides, may be produced by expressing the DNA sequences provided in the invention. Using methods known in the art, a peptide or polypeptide expression vector may be constructed, transformed into a suitable host cell, and conditions suitable for expression of a peptide or polypeptide by the host cell established. One skilled in the art will appreciate that there are a wide variety of suitable vectors for expression in bacterial cells (e.g. pET series (Novagen, Madison, Wis.)), insect cells (e.g. pBlueBac (Invitrogen, Carlsbad, Calif.)), and eukaryotic cells (e.g. pCDNA and pEBVHis (Invitrogen, Carlsbad, Calif.)). In certain embodiments, the caspase-9 or related nucleic acid molecule may be cloned into a gene targeting vector such as pMClneo (Stratagene, La Jolla, Calif.). Synthetic chemistry methods, such as solid phase peptide synthesis can also be used to synthesize proteins, fusion proteins, or polypeptides of the invention.

The resulting expressed peptide or polypeptide can be purified from the culture medium or from extracts of the cultured cells. Methods of protein purification such as affinity chromatography, ionic exchange chromatography, HPLC, size exclusion chromatography, ammonium sulfate crystallization, electrofocusing, or preparative gel electrophoresis are well known and widely used in the art (see generally Ausubel et al., supra; Sambrook et al., supra). An isolated purified protein is generally evidenced as a single band on an SDS-PAGE gel stained with Coomassie blue.

D. Caspase-9 XIAP-Binding Motif Specific Antibodies

Antibodies to the caspase-9 peptides and polypeptides of the invention, and functional variants and functional equivalents of each, are provided by the invention. Antibodies of the invention can be used for a variety of purposes, including research, production and purification, and therapeutic-related purposes. For example, antibodies that specifically bind to caspase-9 peptides, polypeptides, variants, or functional equivalents, can be used to detect the presence of these peptides and polypeptides in a sample. The antibodies can be also used for isolation of corresponding peptides, polypeptides, variants, and functional equivalents and in the identification of molecules that interact with these peptides, polypeptides, variants and functional equivalents. The antibodies may also be used to inhibit or enhance a biological activity of caspase-9 peptides or polypeptides, for example. Thus, the antibodies may also be used therapeutically to inhibit or promote apoptosis of a target cell.

Antibodies of the invention may be used to both directly and indirectly modulate the functional activities of native cellular proteins and recombinantly expressed peptides and polypeptides. One preferred biological activity that may be modulated by antibodies of the invention is the binding of a peptide, polypeptide, functional variant, or functional equivalent to at least a portion of an IAP or to a full length IAP. Antibodies of the invention may be used to inhibit or enhance binding to at least a portion of an IAP. Preferably, this portion of an IAP includes at least one of the BIR domains, i.e. BIR1, BIR2, or BIR3. Accordingly, the antibodies can be specific for the N-terminus of either a caspase-9-p12 or a caspase-9-p10 subunit. In addition, antibodies of the invention may specifically bind a peptide with the consensus amino acid sequence set forth in SEQ ID NO:13. Where an antibody binds to such a consensus IAP-binding motif, it may also bind to other peptides that also share the consensus IAP-binding motif. Without wishing to be bound to any particular proposed theory or mechanism by which an antibody of the invention may inhibit or enhance binding of a caspase-9 peptide or polypeptide to at least a portion of an IAP, an antibody that specifically binds to the IAP-binding motif at the N-terminus of a caspase-9-p12 could sterically hinder subsequent binding to the same region by an IAP. Similarly, an antibody that specifically binds to the IAP-binding motif of Smac/DIABLO could block binding of Smac/DIABLO to an IAP, thus releasing more unbound IAP that can subsequently bind to a caspase-9 IAP-binding motif. In one embodiment, an inhibiting antibody would be specific to an epitope on the N-terminus of a caspase-9-p12 that includes the amino acids (residues 316-319 of SEQ ID NO:1). In another embodiment, an inhibiting antibody would be specific to an epitope of the N-terminus of a caspase-9-plO that includes at least the amino acids Ala-Ile and preferably the amino acids (residues 331-334 of SEQ ID NO:1). In certain embodiments, an antibody that enhanced caspase-9 binding to an IAP would be specific for an eptiope of Smac/DIABLO that includes at least the amino acids (residues 1-4 of SEQ ID NO:8).

Another preferred biological activity that may be modulated by antibodies of the invention is cysteine protease activity. Preferably, such cysteine protease activity is associated with a caspase. Preferably, the caspase is caspase-9 or caspase-3. Without wishing to be bound to any particular theory, an antibody that inhibits IAP binding to a caspase-9 could interfere with IAP inhibition of caspase-9 cysteine protease activity, resulting in enhanced caspase-9 cysteine protease activity. In contrast, an antibody that interfered with IAP binding to a Smac/DIABLO or Omi could result in increase IAP binding to and inhibition of a caspase-9, resulting in decreased caspase-9 cysteine protease activity.

Within the context of the current invention, an antibody includes both polyclonal and monoclonal antibodies (mAb). In addition, an antibody may include fragments generated from any species, including humanized, PrimatizedTm, primate, murine; mouse-human, mouse-primate, and chimeric antibodies. An antibody may be an intact molecule, a fragment thereof (such as scFv, Fv, Fd, Fab, Fab′, and F(ab)′₂ fragments), or multimers or aggregates of intact molecules and/or fragments. An antibody may occur in nature or be produced, e.g., by immunization, synthesis, or genetic engineering. An “antibody fragment,” as used herein, refers to fragments derived from or related to an antibody, which bind antigen and which in some embodiments may be derivatized to exhibit structural features that facilitate clearance and uptake, e.g., by the incorporation of galactose residues. This includes, e.g., F(ab), F(ab)′₂, scFv, light chain variable region (VL), heavy chain variable region (VH), and combinations thereof.

Antibodies may be produced by any of a variety of methods available to one of ordinary skill in the art. Detailed methods for generating antibodies are provided in Antibodies: A Laboratory Manual, Harlow and Lane (eds.), Cold Spring Harbor Laboratories, 1988, which is incorporated by reference. Antibodies are generally accepted as specific for a peptide if they bind with a K_{d} of greater than or equal to 10⁻⁷M, and preferably 10⁻⁸M. The affinity of an antibody can be readily determined by one of ordinary skill in the art (see Skatchard, Ann. N.Y. Acad. Sci. 51:660-672, 1949).

A polyclonal antibody may be readily generated in a variety of animals such as rabbits, mice, and rats. Generally, an animal is immunized with a peptide or one or more peptides comprising caspase-9 or SEQ ID NO:13 amino acid sequences. The peptide may be conjugated to a carrier protein. Routes of administration include intraperitoneal, intramuscular, intraocular, or subcutaneous injections, usually in an adjuvant (e.g, Freund's complete or incomplete adjuvant).

Monoclonal antibodies may be readily generated from hybridoma cell lines using conventional techniques (see Antibodies: A Laboratory Manual, Harlow and Lane (eds.), Cold Spring Harbor Laboratories, 1988). Various immortalization techniques such as those mediated by Epstein-Barr virus or fusion to produce a hybridoma may be used. In a preferred embodiment, immortalization occurs by fusion with a myeloma cell line (e.g., NS-1 (ATCC No. TIB 18) and P3×63—Ag 8.653 (ATCC No. CRL 1580)) to create a hybridoma that secretes a monoclonal antibody.

Antibody fragments, such as Fab and Fv fragments, may be constructed, for example, by conventional enzymatic digestion or recombinant DNA techniques to yield isolated variable regions of the antibody. Within one embodiment, the genes which encode the variable region from a hybridoma producing a monoclonal antibody of interest are amplified using nucleotide primers corresponding to the variable region. Amplification products are subcloned into plasmid vectors and propagated and purified using bacteria, yeast, plant or mammalian-based expression systems. Techniques may be used to change a murine antibody to a human antibody, known familiarly as a “humanized” antibody, without altering the binding specificity of the antibody.

Antibodies may be assayed for immunoreactivity against peptides and polypeptides comprising amino acid sequences corresponding to a caspase-9 or SEQ ID NO:13 by any of a number of methods, including western blot, enzyme-linked immuno-sorbent assays (ELISA), countercurrent immuno-electrophoresis, radioimmunoassays, dot blot assays, sandwich assays, inhibition or competition assays, and immunoprecipitation (see U.S. Pat. Nos. 4,376,110 and 4,486,530; see also Antibodies: A Laboratory Manual, Harlow and Lane (eds.), Cold Spring Harbor Laboratory Press, 1988). Techniques for purifying antibodies are available in the art. In certain embodiments, antibodies are purified by passing the antibodies over an affinity column to which amino acid sequences of the present invention are bound. Bound antibody is then eluted. Other purification techniques include, but are not limited to HPLC or RP-HPLC and purification on protein A or protein G columns.

A number of therapeutically useful molecules are known in the art that comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment that comprises both antigen-binding sites. An “Fv” fragment can be produced by preferential proteolytic cleavage of an IgM or, on rare occasions, an IgG or an IgA immunoglobulin molecule. Fv fragments are more commonly derived using recombinant techniques known in the art. The Fv fragment includes a non-covalent V_{H}::V_{L} heterodimer, including an antigen-binding site that retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated, but chemically separated, light and heavy polypeptide chains from an antibody V region into an sFv molecule that will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set that provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs that form an antigen-binding surface. It is generally recognized that there are conserved structural regions of FRs that influence the folded shape of the CDR loops into certain “canonical” structures—regardless of the precise CDR amino acid sequence. Further, certain FR residues are known to participate in non-covalent interdomain contacts that stabilize the interaction of the antibody heavy and light chains.

A “humanized” antibody refers to an antibody derived from a non-human antibody (typically murine) or derived from a chimeric antibody, which retains or substantially retains the antigen-binding properties of the parent antibody but which is less immunogenic in humans. This may be achieved by various methods, including, for example: (a) grafting only the non-human CDRs onto human framework and constant regions (humanization); or (b) transplanting the entire non-human variable domains, but “cloaking” them with a human-like surface by replacement of surface residues (“veneering”). Such methods are disclosed, for example, in Jones et al., Nature 321:522-525, 1986; Morrison et al., Proc. Natl. Acad. Sci. 81:6851-6855, 1984; Morrison and Oi, Adv. Immunol. 44:65-92, 1988; Verhoeyer et al., Science 239:1534-1536, 1988; Padlan, Molec. Immun. 28:489-498, 1991; Padlan, Molec. Immun. 31(3):169-217, 1994. In the present invention, humanized antibodies include “humanized” and “veneered” antibodies. A preferred method of humanization comprises the alignment of the non-human heavy and light chain sequences to human heavy and light chain sequences, selection and replacement of the non-human framework with a human framework based on such alignment, molecular modeling to predict conformation of the humanized sequence, and comparison to the conformation of the parent antibody, followed by repeated back mutation of residues in the CDR region that disturb the structure of the CDRs, until the predicted conformation of the humanized sequence model closely approximates the conformation of the non-human CDRs of the parent non-human antibody.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues that are readily encountered by the immune system are selectively replaced with human residues to provide a hybrid molecule that comprises either a weakly immunogenic, or substantially non-immunogenic veneered surface. The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments.

There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR that differ from the human counterpart are replaced by the residues present in the human moiety, using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids. In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g, electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

E. Methods of Using SMAC Nucleic Acids and Peptides or Polypeptides

Caspase-9 is a key component of caspase-mediated apoptosis. Studies using caspase-9 peptides and polypeptides of the present invention revealed that IAP's could inhibit caspase-mediated apoptosis by binding to caspase-9, thereby inhibiting its cysteine protease functional activity. The ability and availability of IAP's to bind and inhibit caspase-9 is regulated by other IAP-binding proteins, including, for example, Smac/DIABLO and Omi. These proteins appear to compete with caspase-9 for binding to IAP's. Thus, caspase-9-mediated apoptosis appear to be regulated by multiple different protein:protein interactions and is governed, at least in part, by the predominant IAP complexes formed within a cell. The nucleic acids, peptides, polypeptides, and antibodies of the invention can be used to alter IAP's ability to bind caspase-9. Thus, these compounds, and compositions comprising these compounds, can be used to alter apoptosis within a cell. In addition, nucleic acids, peptides, polypeptides, antibodies, and compositions thereof, may be used to identify other modulators of apoptosis, including both enhancers and inhibitors. Thus, the compositions described herein, including caspase-9 nucleic acids, peptides, polypeptides, and antibodies, can be used for a variety of assays and for therapeutic purposes.

1. Identification of Inhibitors and Enhancers of Caspase-Mediated Apoptotic Activity

Inhibitors and enhancers of apoptosis can be used for a variety of purposes, including therapeutically. For example, inhibitors and enhancers of apoptosis may be used to treat cells displaying aberrant levels of apoptosis. More specifically, inhibitors may be used to treat cells displaying greater than desirable levels of apoptosis, while enhancers may be used to treat cells displaying less than desirable levels of apoptosis. Inhibitors of apoptosis are particularly useful for treating pathologies associated with inappropriate activation of apoptosis, such as AIDS, neurodegenerative disease, and ischemic injury. Enhancers of apoptotic activity are desirable for treating pathologies associated with a loss of apoptosis, such as tumors or cells that mediate autoimmune diseases. Enhancers of apoptosis may also be used to destroy targeted tissues, if desired. Similarly, inhibitors of apoptosis may also be used to preserve targeted cells and tissues, if desired. Targeted cells and tissues do not necessarily display aberrant levels of apoptosis. Rather, such cells and tissues may be targeted because they possess other harmful or beneficial characteristics.

Inhibitors and enhancers of apoptosis may act through a wide variety of mechanisms. Certain of these mechanisms involve IAP binding to caspase-9 proteins. Without wishing to be bound to a particular theory or held to a particular mechanism, an enhancer may act by interfering with caspase-9 binding to an IAP, or by other mechanisms. Similarly, an inhibitor may act by stabilizing or enhancing caspase-9 binding to an IAP. An inhibitor may act directly or indirectly. For example, an enhancer may indirectly activate caspase-9-mediated apoptosis by itself binding to an IAP, or by increasing or stabilizing IAP binding to another molecule, such as Smac/DIABLO. An inhibitor may indirectly prevent caspase-9-mediated apoptosis by interfering with IAP binding to Smac, thereby promoting IAP binding to and inhibition of caspase-9. Enhancers may also increase the rate or efficiency of caspase processing, increase transcription or translation, decrease proteolysis, or act through other mechanisms. Generally, inhibitors may act through opposing mechanisms.

Candidate inhibitors and enhancers include small molecules (organic molecules), nucleic acids, peptides, and polypeptides. Inhibitors should have a minimum of side effects and are preferably non-toxic. Candidate inhibitors and enhancers may be isolated or procured from a variety of sources, such as bacteria, fungi, plants, parasites, and libraries of chemicals, peptides, or peptide derivatives, for example. Inhibitors and enhancers may be also rationally designed, based on protein structures determined from X-ray crystallography. Within the context of the present invention, caspase-9 peptides, polypeptides, nucleic acids, antibodies, and functional variants and functional equivalents of each, can act as inhibitors or enhancers. In certain embodiments, the caspase-9 nucleic acids, antibodies, peptides, polypeptides, and functional variants and functional equivalents of each, can be used as promoters of caspase enzymatic activity at attainable concentrations to kill cancer cells that overexpress IAPs or as components in a chemotherapy regimen to sensitize cancers. Preferably, caspase-9 molecules of the invention that are capable of binding to at least a portion of an IAP but lack cysteine protease activity can enhance apoptosis by competing with endogenous caspase-9 for IAP binding. Preferably, caspase-9 molecules of the invention that fail to undergo normal proteolytic processing and do not bind to at least a portion of an IAP can enhance apoptosis, since they are not inhibited by endogenous IAP's.

Screening assays for inhibitors and enhancers will vary according to the type of inhibitor or enhancer and the nature of the activity that is being affected. Assays may be performed in vitro or in vivo. In general, assays are designed to evaluate apoptotic pathway activation or inhibition (e.g., caspase protein processing, caspase enzymatic activity, cell morphology changes, DNA laddering, cell viability, and the like). In any of the assays, a statistically significant increase or decrease compared to a proper control is indicative of enhancement or inhibition. In certain embodiments, screening assays examine the activity of a specific caspase. Preferably, this caspase is selected from the group consisting of caspase-3, caspase-7, and caspase-9.

In certain embodiments, methods of identifying an inhibitor or enhancer of a caspase-mediated apoptosis involve contacting a cell expressing a caspase-9 peptide or polypeptide, or a variant or derivative thereof, with a candidate inhibitor or enhancer, and evaluating apoptotic pathway activation or inhibition. In one preferred embodiment, the cell contains a vector expressing a peptide or polypeptide comprising at least an amino acid sequence set forth in SEQ ID NO:13 and capable of binding to at least a portion of an IAP. For example, this peptide or polypeptide may comprise the N-terminal four amino acid residues of either caspase-9-p12 set forth in SEQ ID NO:6, Smac/DIABLO set forth in SEQ ID NO:8, or Omi set forth in SEQ ID NO:9. In other embodiments, this peptide or polypeptide may comprise the caspase-9 linker peptide set forth in SEQ ID NO:11 or the Smac-N7 peptide set forth in SEQ ID NO:12. In another preferred embodiment, the cell contains a vector expressing a peptide or polypeptide comprising at lest a mutated procaspase-9, or variant thereof, that fails to under go normal processing. Examples of such mutant procaspase-9 polypeptides include the previously described single, double, and triple mutants, as well as a linker region deletion mutant.

The effect of a candidate inhibitor or enhancer can be determined by any known experimental procedure or method that is capable of measuring apoptosis. For example, an increase in cell viability compared to a control indicates the presence of an inhibitor and a decrease in cell viability as compared to a control indicates the presence of an enhancer. Cell viability can be determined by any means known in the art, including trypan blue exclusion staining. The effect of a candidate can also be determined by directly examining caspase processing or enzymatic activity, wherein increased processing or enzymatic activity as compared to a control indicate an enhancer, while decreased processing or enzymatic activity indicate an inhibitor. One method of examining processing activity is to directly examine the presence of large and small caspase subunits. Preferably, these are caspase-3, caspase-9, or caspase-7 subunits. One method of determining caspase enzymatic activity is to detect the presence of substrate cleavage products. Preferably, the activity being measured is the enzymatic activity of a caspase-3, caspase-7, or caspase-9. A preferred substrate is acetyl DEVD-aminomethyl coumarin.

One preferred in vitro assay is performed by examining the effect of a candidate compound on the activation of an initiator caspase (e.g., caspase-9) or an effector caspase (e.g., caspases 3-7). Briefly, procaspase-9, an IAP, cytochrome c, dATP and a caspase-9 peptide or polypeptide, or a variant or derivative thereof, are provided. The processing of caspase-9 into two subunits can be assayed, or, alternatively, caspase-9 enzymatic activity can be monitored by adding procaspase-3, procaspase-7, or other effector caspases and monitoring the activation of these caspases either directly via subunit formation or via substrate cleavage (e.g., acetyl DEVD-aminomethyl coumarin (amc), lamin, PRPP, PARP, and the like). Further, to facilitate detection, typically the protein of interest may be in vitro translated and labeled during translation. This composition is incubated with a caspase-9 peptide, polypeptide, functional variant or functional equivalent, in the presence or absence of a candidate inhibitor or enhancer. Processing of caspase-9 into two subunits can be monitored, as can processing/activation of a coincubated effector pro-caspase. Caspase processing is routinely monitored either by gel electrophoresis or indirectly by monitoring caspase substrate turnover. The two subunits and caspase substrate turnover may be readily detected by autoradiography after gel electrophoresis. One skilled in the art will recognize that other methods of labeling and detection may be used alternatively.

Another means of identifying an inhibitor or enhancer of apoptosis involves identifying a compound that inhibits or enhances the binding of a caspase-9 peptide or polypeptide, or a variant or derivative thereof, to at least a portion of an IAP. Preferably, the caspase-9 peptide or polypeptide contains an amino acid sequence set forth in SEQ ID NO:13 and is capable of binding to at least a portion of an IAP. The ability of a compound to disrupt or enhance binding can be determined by any means available, including examining the effect of the compound on in vitro binding of the peptide to at least a portion of an IAP, preferably a BIR1, BIR2, or BIR3 domain, or a full length IAP. Alternatively, a functional assay may be performed to examine displacement of the peptide or polypeptide from binding at least a portion of an IAP. Preferred functional assay determine caspase processing and enzymatic activity.

Moreover, any known enzymatic analysis can be used to follow the inhibitory or enhancing ability of a candidate compound with regard to the ability of caspase-9 molecules, or variants thereof, of the present invention to promote or inhibit the enzymatic activity of caspases. For example, one could express a caspase-9 construct of interest in a cell line, be it bacterial, insect, mammalian or other, and purify the resulting polypeptide. The purified caspase-9 peptide or polypeptide can then be used in a variety of assays to follow its ability to promote the enzymatic activity of effector caspases or apoptotic activity. Such methods of expressing and purifying recombinant proteins are known in the art and examples can be found in Sambrook et al., Molecular Cloning: A Laboratory Manual, Cold Spring Harbor Press, 1989 as well as in a number of other sources.

In vivo assays are typically performed in cells transfected either transiently or stably with an expression vector containing caspase-9 nucleic acid molecule such as those described herein. These cells are used to measure caspase processing, caspase substrate turnover, enzymatic activity of effector caspases or apoptosis in the presence or absence of a candidate compound. When assaying apoptosis, a variety of cell analyses may be used including, for example, dye staining and microscopy to examine nucleic acid fragmentation, porosity of the cells, and membrane blebbing.

A variety of other methodologies exist that can be used to investigate the effect of a candidate compound. Such methodologies are those commonly used to analyze enzymatic reactions and include, for example, SDS-PAGE, spectroscopy, HPLC analysis, autoradiography, chemiluminescence, chromogenic reactions, and immunochemistry (e.g., blotting, precipitating, etc.).

2. Compositions and Methods of Modulating Apoptosis

Compositions comprising a caspase-9 peptide, polypeptide, nucleic acid, or antibody, or a variant or derivative of any of these, are provided by the invention. In addition, other compositions of the invention may comprise an inhibitor or enhancer of apoptosis or IAP binding identified by a method of the invention. Compositions of the invention may potentially be used for a variety of purposes, but they are preferably used to inhibit or promote apoptosis. Preferably, compositions of the invention are used in methods of inducing or stimulating apoptosis, such methods also being provided by the invention. These methods can be used to induce apoptosis of a target cell, such as, for example, a neoplastic or tumor cell. Thus, compositions of the invention preferably also contain a physiologically acceptable carrier. The term “physiologically acceptable carrier” refers to a carrier for administration of a first component of the composition which is selected from antibodies, peptides or nucleic acids. Suitable carriers and physiologically acceptable salts are well known to those of ordinary skill in the art. A thorough discussion of acceptable carriers is available in Remington's Pharmaceutical Sciences, Mack Publishing Co., NJ, 1991).

Polynucleotide compositions include mammalian expression vectors, sense RNAs, ribozymes, and antisense RNA, for example. Expression vectors and sense RNA molecules are designed to express caspase-9 fragments or variants thereof, while ribozymes and antisense RNA constructs are designed to reduce the levels of caspase-9 polypeptides expressed. Preferred nucleic acid compositions comprise polynucleotides capable of expressing peptides and polypeptides that are capable of binding to at least a portion of an IAP. For example, these peptides and polypeptides include peptides and polypeptides comprising the consensus IAP-binding motif set forth in SEQ ID NO:13 and capable of binding to at least a portion of an IAP. Other preferred nucleic acid compositions comprise polynucleotides capable of expressing mutant procaspase-9 polypeptides, or variants thereof, that fail to undergo normal proteolytic processing, such as the single mutant, double mutant, triple mutant, and linker deletion mutants described herein. Furthermore, nucleic acid compositions include any and all compositions comprising an expression vector provided by the invention.

Peptide and polypeptide compositions include peptides or polypeptides that are capable of binding to at least a portion of an IAP, including those containing a peptide sequence identified in SEQ ID NO:13 and capable of binding to at least a portion of an IAP. Other preferred polypeptide compositions of the invention include mutant procaspase-9 polypeptides, or variants thereof, that fail to undergo normal proteolytic processing, such as the single mutant, double mutant, triple mutant, and linker deletion mutants described herein. Furthermore, peptide and polypeptide compositions include any and all compositions comprising any peptide or polypeptide provided by the invention.

Antibody compositions include, but are not limited to, polyclonal, monoclonal, single chain and humanized antibodies and antibody fragments. These compositions may comprise, for example, polyclonal antibodies that recognize one or more epitopes of caspase-9, particularly epitopes including the N-terminal IAP binding region of caspase-9-p12. Thus, in one embodiment, an antibody recognizes an epitope that includes the amino acids (residues 316-319 of SEQ ID NO:1). However, antibody compositions are not limited to those containing antibodies capable of binding to caspase-9. Antibody compositions also include those containing antibodies that specifically bind to an epitope comprising at least one of the amino acid sequences disclosed in SEQ ID NO:13. The antibodies of the composition may recognize native and/or denatured peptides and polypeptides, such as caspase-9. These antibodies may be produced according to methods well known in the art, as described above.

Other compound compositions of the invention include inhibitors or enhancers of apoptosis. Preferably, such inhibitors or enhancers are identified using methods provided by the invention. Inhibitors and enhancers include, but are not limited to, small molecules (organic molecules), peptides, polypeptides, and nucleic acids.

Appropriate dosage amounts balancing toxicity and efficacy will be determined during any clinic testing pursued, but a typical dosage will be from about 0.001 mg/kg to 50 mg/kg or 0.05 mg/kg to about 10 mg/kg of the polynucleotide, peptide or antibody. If used in gene therapies such dosages will depend on the vector utilized and will be determined during any clinic testing pursued Compositions of the invention may be used to stimulate or induce apoptosis in a cell, including a cell that overexpresses an IAP and neoplastic or tumor cells. Indeed, the invention provides methods of using the compounds and compositions of the invention to induce or stimulate apoptosis of a cell. Preferably, such methods comprise contacting a cell with a nucleic acid, peptide, polypeptide, antibody, or inhibitor or enhancer of the invention, under conditions and for a time sufficient to permit induction of apoptosis in the cell.

The compositions of the invention can be (1) administered directly to the subject; (2) delivered ex vivo to cells derived from the subject; or (3) delivered in vitro. Direct delivery will generally be accomplished by injection. Alternatively, compositions can also be delivered via oral or pulmonary administration, suppositories, transdermally, or by gene guns, for example. Dosage treatment may be a single dose or multiple doses.

Methods of ex vivo delivery and reimplantation of transformed cells into a subject are known in the art. Generally, delivery of nucleic acids for both ex vivo and in vitro applications can be accomplished by, for example, dextran-mediated transfection, calcium phosphate precipitation transfection, viral infection, polybrene mediated transfection, protoplast fusion, electroporation, encapsulation of polynucleotides in liposomes, and direct microinjection of the DNA into nuclei, all well known in the art.

Gene therapy vectors comprising caspase-9 nucleic acid sequences, or complements or variants thereof, are within the scope of the invention. These vectors may be used to regulate mRNA and peptide or polypeptide expression in target cells. In some instances, it may be advantageous to increase the amount of caspase-9 nucleic acids or caspase-9 polypeptides that are expressed. In other cases, gene therapy vectors may be used to decrease functional caspase-9 levels. Gene therapy vectors may comprise any caspase-9 nucleic acid of the current invention, including fragments, variants, antisense, ribozymes, and mutants. Additionally, gene therapy vectors may express any caspase-9 peptide or polypeptide, including fragments, variants, and mutants. Gene therapy vectors may also express inhibitors or enhancers of apoptosis. Expression of nucleic acids may be controlled by endogenous mammalian or heterologous promoters, and it may be either constitutive or regulated. Nucleic acids used according to the invention may be stably integrated into the genome of the cell or may be maintained in the cell as extra-nuclear or episomal DNA. It some circumstances, it may be preferable for the expression vector to direct tissue-specific expression of the encoded nucleic acid or polypeptide.

Caspase-9 and other nucleic acid molecules may be delivered by any method of gene delivery available in the art. Gene delivery vehicle may be of viral or non-viral origin (see generally Jolly, Cancer Gene Therapy 1:51-64, 1994; Kimura, Human Gene Therapy 5:845-852, 1994; Connelly, Human Gene Therapy 1:185-193, 1995; and Kaplitt, Nature Genetics 6:148-153, 1994). The present invention can employ recombinant retroviruses that are constructed to carry or express a nucleic acid molecule of the invention. Methods of producing recombinant retroviral virions suitable for gene therapy have been extensively described (see, e.g., Mann et al. Cell 33:153-159, 1983; Nikolas and Rubenstein, Vectors: A survey of molecular cloning vectors and their uses, Rodriquez and Denhardt (eds.), Stoneham:Butterworth, 494-513, 1988). The present invention also employs viruses such as alphavirus-based vectors, adenovirus, and parvovirus that can function as gene delivery vehicles. Examples of vectors utilized by the invention include intact adenovirus, replication-defective adenovirus vectors requiring a helper plasmid or virus, and adenovirus vectors with their native tropism modified or ablated such as adenoviral vectors containing a targeting ligand. Other examples include adeno-associated virus based vectors and lentivirus vectors.

Packaging cell lines suitable for use with the above-described viral and retroviral vector constructs may be readily prepared and used to create producer cell lines (also termed vector cell lines) for the production of recombinant vector particles.

Examples of non-viral methods of gene delivery vehicles and methods which may be employed according to the invention include liposomes (see, e.g., Wang et al. PNAS 84:7851-7855, 1987), polycationic condensed DNA (see, e.g., Curiel, Hum. Gene Ther. 3:147-154, 1992); ligand linked DNA (see, e.g., Wu, J. Biol. Chem. 264:16985-16987, 1989); deposition of photopolymerized hydrogel materials; hand-held gene transfer particle guns, as described in U.S. Pat. No. 5,149,655; ionizing radiation as described in U.S. Pat. No. 5,206,152 and WO 92/11033; and nucleic charge neutralization or fusion with cell membranes. Additional approaches are described in Philip, Mol. Cell Biol. 14:2411-2418, 1994 and in Woffendin, Proc. Natl. Acad. Sci. 91:1581-1585, 1994. Conjugates comprising a receptor-binding internalized ligand capable of delivering nucleic acids may also be used according to the present invention. Conjugate-based preparations and methods of use thereof are described in WO 96/36362 which is hereby incorporated by reference in its entirety. Other non-viral delivery methods include, but are not limited to, mechanical delivery systems such as the approach described in Woffendin et al., Proc. Natl. Acad. Sci. USA 91(24):11581-11585, 1994 and naked DNA protocols. Exemplary naked DNA introduction methods are described in WO 90/11092 and U.S. Pat. No. 5,580,859.

In other embodiments, methods of the invention utilize bacteriophage delivery systems capable of transfecting eukaryotic cells. Bacteriophage-mediated gene transfer systems are described in WO 99/10014, which is incorporated in its entirety. Phage delivery vehicles may express a targeting ligand on their surface that facilitates receptor-mediated gene delivery.

In addition, compositions and methods of modulating apoptosis using small molecule agonists or antagonists or heterologous polypeptides that bind to a caspase-9 peptide or polypeptide, or a variant of derivative thereof, are included within the scope of the current invention.

3. Methods of Manufacturing Inhibitors and Enhancer of Apoptosis

Compounds that inhibit or enhance apoptosis may be produced and manufactured by any means available in the art. Generally, the particular method of producing a compound of the invention will depend upon the biological characteristics of the molecule, such as whether it is a peptide, nucleic acid, antibody, small molecule, or another type of molecule. Methods of producing various types of biological and chemical compounds are widely known in the art.

A preferred method of producing a compound for inhibiting or enhancing apoptosis involves identifying an inhibitor or enhancer according to a method of the invention and purifying the inhibitor or enhancer. A preferred process for manufacturing a compound that inhibits or enhances apoptosis includes identifying such an inhibitor or enhancer and derivitizing the compound. Optionally, derivitized compounds may be further identified as inhibitors or enhancers of apoptosis according to a method provided by the invention and/or further derivitized to produce a compound that inhibits or enhances apoptosis.

### EXAMPLES

The following experimental examples are offered by way of illustration, not limitation.

### Example 1

### Fully Processed and Unprocessed Procaspase-9 are Catalytically Active

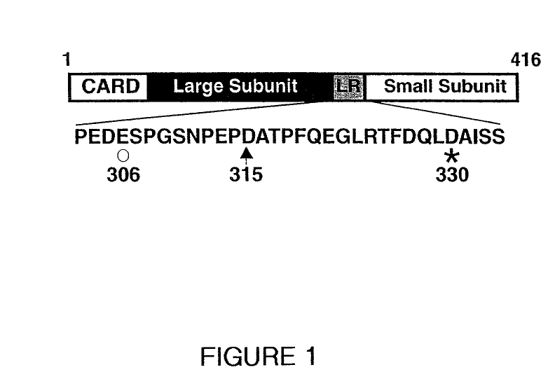

This example discloses that the X-linked inhibitor of apoptosis protein (XIAP) associates with the active Apaf-1-caspase-9 holoenzyme complex through binding to the N-terminus of the linker peptide on the small subunit of caspase-9, which becomes exposed after proteolytic processing of procaspase-9 at Asp315 (see FIG. 1).

Data suggested that processing of procaspase-9 might be required for inhibition by XIAP, since the overexpression of XIAP was not able to inhibit DNA damage-induced processing of procaspase-9 in U-937 cells, but inhibited the catalytic activity of processed caspase-9 (Datta, R. et al., J. Biol Chem 275:31733-31738, 2000). To understand the mechanism of inhibition of the caspase-9-Apaf-1 holoenzyme complex, in vitro Apaf-1-caspase-9 holoenzyme complexes containing either fully processed caspase-9 or unprocessed procaspase-9 were reconstituted and their catalytic activity was examined.

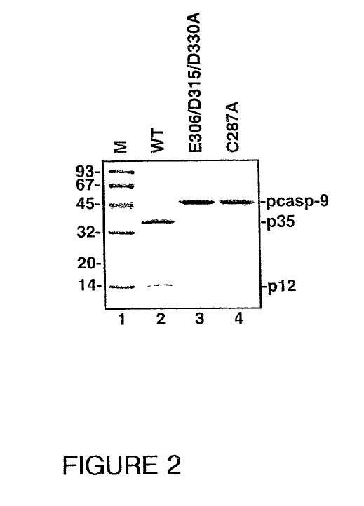

To produce fully processed caspase-9, wild-type (WT) procaspase-9 was overexpressed in Escherichia coli strain BL21 (DE3) as a C-terminally 6-Histidine-tagged protein using the pET-21c or pET-28a vector (Novagen), which resulted in complete processing of procaspase-9 into its p35 and p12 subunits (FIG. 2, lane 2). Sequence analysis of the purified recombinant caspase-9 revealed that greater than 90% of caspase-9 processing in bacteria occurred at Asp3l5, which generated the p35 and p12 subunits, and the remaining 10% of processing occurred at Asp330 to generate a p10 subunit. A minor processing was also detected at Glu306. To produce a recombinant unprocessed procaspase-9, Asp315, Asp330, and Glu306 were mutated to Ala. Expression was confirmed by Coomassie staining of SDS-PAGE resolved proteins (FIG. 2). Overexpression of the triple mutant procaspase-9 (E306/D315/D330A) produced an unprocessed protein (FIG. 2, lane 3).

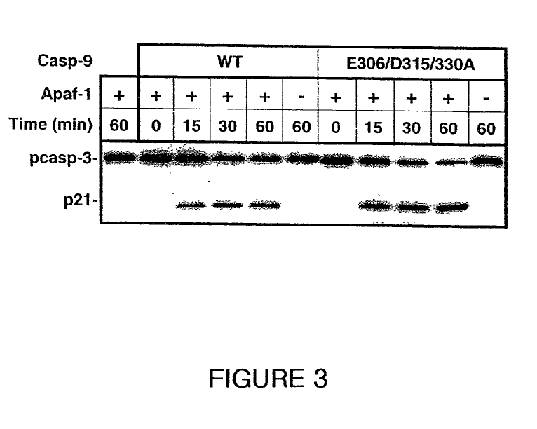

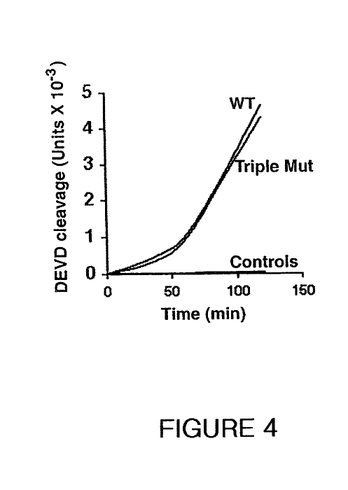

When the processed WT caspase-9 or the triple mutant procaspase-9 proteins were reconstituted with purified Apaf-1 at physiological concentrations of 20 nM each, the triple mutant procaspase-9 was as efficient as the fully processed WT caspase-9 in processing procaspase-3 C163A, or inducing DEVD-aminomethyl coumarin (DEVD-AMC) cleavage in Apaf-1-caspase-9-deficient S100 fraction in the presence of Apaf-1, cytochrome c, and dATP, but not in their absence (FIGS. 3 and 4).

Procaspase-3 processing assays were generally performed as described in Srinivasula, S. M. et al. J Biol Chem 275:36152-36157, 2000. In this assay, purified recombinant procaspase-3 C163A was incubated with equal amounts of recombinant WT or triple mutant caspase-9 protein (20 nM) in the presence (+) or absence (−) of recombinant Apaf-1 (20 nM). The reaction mixtures were stimulated with cytochrome c (50 ng/μl) and dATP (1 mM), incubated for 0-60 minutes at 30° C., and then analyzed by SDS-PAGE and western blot analysis (FIG. 3).

Caspase-3 enzymatic assays with the tetrapeptide substrate DEVD-AMC were generally performed as described in Srinivasula, S. M. et al. J Biol Chem 275:36152-36157, 2000. In this caspase-3 enzymatic assay, caspase-9-depleted S100 extracts from Apaf-1-deficient mouse embryonic fibroblasts were incubated with equal amounts of recombinant WT and triple mutant caspase-9 proteins together with Apaf-1, cytochrome c and dATP. The controls used in these assays represent WT and triple mutant caspase-9 proteins incubated as above without cytochrome c and dATP. The reactions were carried out in the presence of 100 μM of DEVD-AMC for 0-120 minutes, and substrate cleavage was measured by luminescence spectrometry using a Perkin Elmer Luminescence spectrometer and represented in arbitrary spectrometric units.

### Example 2

### XIAP Inhibits Only Processed Caspase-9

This example confirms that XIAP does not inhibit activation of procaspase-9, but inhibits the activity of the processed caspase-9.

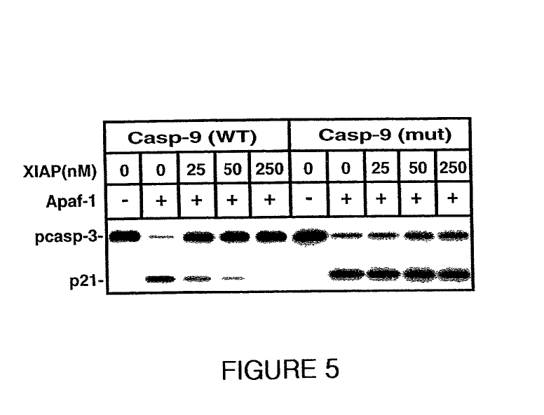

Given that both processed and unprocessed caspase-9-Apaf-1 holoenzyme complexes are catalytically active, it was determined whether XIAP could inhibit them equally. Catalytic activity reactions were carried out by or the uncleavable triple mutant (E306/D315/330A) caspase-9 proteins (specific activity ˜10 fluorogenic units sec⁻¹ ng⁻¹, cytochrome c, and dATP in the presence (+) or absence (−) of Apaf-1 (20 nM). The effect of XIAP was examined by including increasing amounts of XIAP in the reactions. Reaction products were analyzed by SDS-PAGE and autoradiography. As shown in FIG. 5, XIAP did not significantly inhibit the processing of procaspase-3 by the holoenzyme containing the mutant caspase-9, but it completely inhibited the processing by the holoenzyme containing the WT caspase-9.

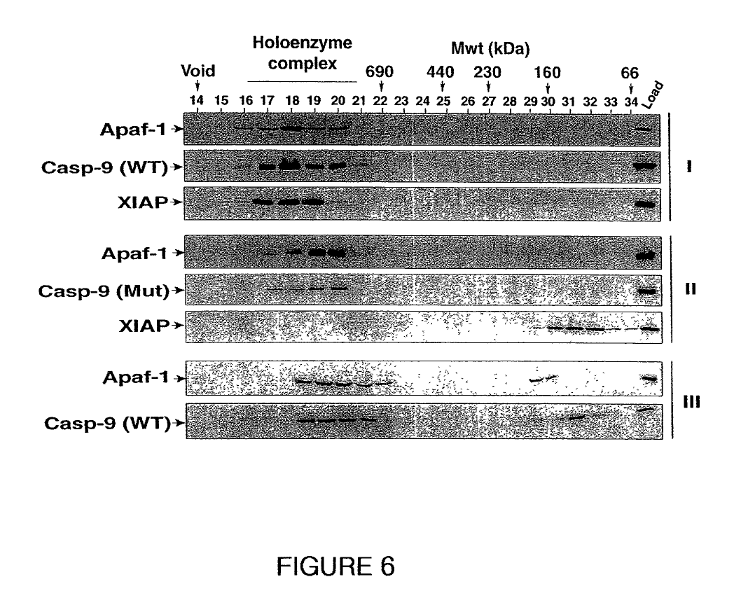

The loss of inhibition of the catalytic activity of the holoenzyme containing the mutant caspase-9 could be due to the inability of XIAP to associate with the uncleavable caspase-9 in the holoenzyme complex. To test this hypothesis, the two complexes were analyzed after incubation with XIAP by gel filtration on a Superose-6 FPLC column. Gel-filtration analysis of the Apaf-1-caspase-9 holoenzyme complex was performed as described in Saleh, A. et al., J Biol Chem 274:17941-17945, 1999. For this analysis, WT or uncleavable caspase-9 were mixed with purified Apaf-1 at equal molar ratios together with cytochrome c (50 ng/μl) and dATP (1 mM), followed by incubation with XIAP (FIG. 6, panels I and II) or nothing (FIG. 6, panel III) at room temperature for one hour in oligomerization buffer 1 (25 mM HEPES (pH 7.4), 50 mM NaCl, 10 mM KCl, 5 mM MgCl₂, 100 μg/ml BSA, 5% glycerol, and 0.1 mM DTT). After incubation, the reaction mixtures were diluted with oligomerization buffer I, and aliquots of each sample (100 μl) were loaded onto a Superose 6 FPLC column. Equal volumes of the column fractions (50 μl) were separated by SDS-PAGE and immunoblotted with anti-Apaf-1, anti caspase-9 or anti-XIAP antibodies.

As shown in FIG. 6, both wild type and uncleavable caspase-9 formed large (˜1.4 mDa) holoenzyme complexes with Apaf-1 after stimulation with cytochrome c and dATP. Interestingly, XIAP co-migrated with the wild type caspase-9-Apaf-1 complex but not with the uncleavable caspase-9-Apaf-1 complex. The size of calibration protein standards and their elution positions from the Superose 6 column are indicated by vertical arrows above the upper panel of FIG. 6.

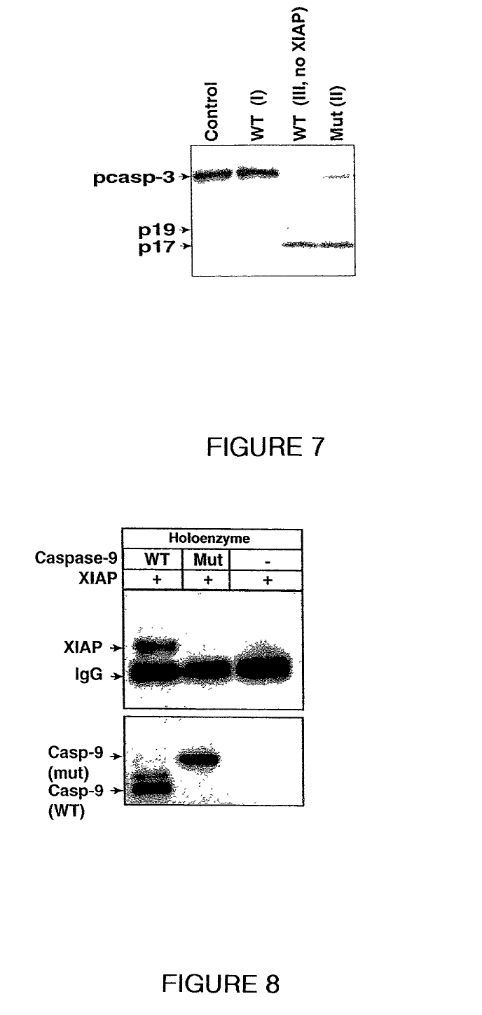

Next, enzymatic activity assays were performed by incubating ³⁵S-labeled procaspase-3 (1 μl) with buffer (control) or equal amounts of aliquots of the peak fractions (40 μl) containing the caspase-9-Apaf-1 holoenzyme complexes from runs I (WT, with XIAP), III (WT, without XIAP) and II (Mut, with XIAP), respectively, for one hour at 30° C. Samples were then analyzed by SDS-PAGE and autoradiography. As shown in FIG. 7, the uncleavable caspase-9-Apaf-1 complex was able to process procaspase-3 (FIG. 7, panel II), whereas the WT caspase-9-Apaf-1-XIAP complex was completely inactive (FIG. 7, panel I). A control WT caspase-9-Apaf-1 complex that was reconstituted without XIAP was fully active (FIG. 7, panel III). This demonstrated that XIAP associated with and inhibited the activity of the WT-caspase-9-Apaf-1 complex, but not the uncleavable caspase-9-Apaf-1 complex. This indicated that processing of caspase-9 at the interdomain linker region is important for binding to XIAP To further confirm the gel filtration data, WT or uncleavable caspase-9-Apaf-1 complexes were assembled by incubation of the caspase-9 variants with purified Apaf-1, cytochrome c, and dATP. The complexes were purified on Superose 6 FPLC column and then incubated with XIAP (50 nM). After incubation, the complexes were immunoprecipitated with an anti-Apaf-1 antibody, fractionated by SDS-PAGE, and immunoblotted with an XIAP antibody (FIG. 8, upper panel) or a caspase-9 antibody (FIG. 8, lower panel). Only the WT caspase-9-Apaf-1 complex contained XIAP (FIG. 8, upper panel). These data were consistent with recent observations that revealed that XIAP did not inhibit activation of procaspase-9 but inhibited the activity of the processed caspase-9 in cells undergoing apoptosis.

### Example 3

### Linker Region of Fully Processed Caspase-9 Binds to the BIR3 Domain of XIAP

This example discloses that cleavage of caspase-9 at Asp315 exposes the XIAP-binding motif in the caspase-9 linker region, thus allowing binding to the BIR3 domain of XIAP and concomitant inhibition of caspase-9 activity.

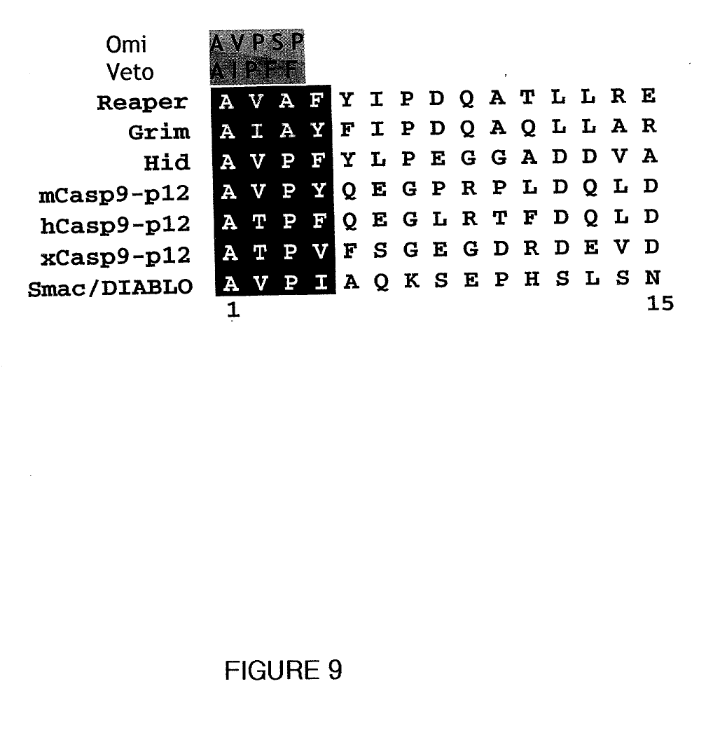

Because the uncleavable caspase-9-Apaf-1 complex is catalytically active, the inability of XIAP to associate with it and inhibit its activity suggested that the association between caspase-9 and XIAP did not require the active site cysteine, but most likely involved residues exposed after autoprocessing of procaspase-9 at Asp3 15. Interestingly, examination of the free N-terminus of the human, mouse and Xenopus p12 subunit of caspase-9, generated after autoprocessing at Asp315¹⁰, revealed that they all contain a 4-residue motif similar to the BIR3-interaction motif present at the N-terminus of mature Smac/DIABLO (see FIG. 9). This motif also has significant homology to the IAP-interaction motif at the N-termini of the Drosophila proteins Hid, Reaper and Grim (see FIG. 9).

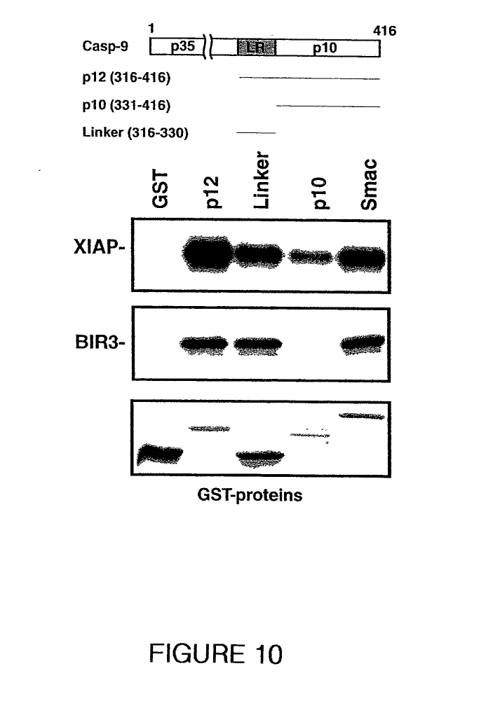

To determine whether this conserved motif interacts with XIAP, in vitro interaction assays were performed with ³⁵S-labeled full length XIAP or the isolated BIR3 domain of XIAP and C-terminal GST fusion proteins of Caspase-9-p12 (residues 316-416 of SEQ ID NO:1), -plO (residues 331-416 of SEQ ID NO:1) or the linker region/peptide PEDESPGSNPEPDATPFQEGLRTFDQLDAISS, (residues 316-330, SEQ ID NO:22) (see FIG. 10). The C-terminal GST fusion proteins were expressed in bacteria and then immobilized onto glutathione-affinity resin. The resin was incubated with in vitro translated ³⁵S-labeled XIAP or the BIR3 domain of XIAP, washed extensively, and then analyzed by SDS-PAGE and autoradiography. The caspase-9 deletion mutants used in these studies are represented by bar diagrams above the panel in FIG. 10.

Interestingly, both p12 and the linker peptide were able to interact with full length XIAP as well as with the isolated BIR3 domain of XIAP. The p10-GST fusion protein was also able to interact, but only weakly (˜50 to 100-fold less), with the full length XIAP or the BIR3 domain. This weak interaction was due to the conservation of the first two residues of the BIR3-binding motif on the N-terminus of p10 (human, Ala331-Ile332; mouse, Ala331-Val332), since single point mutation of these two residues completely abolished the weak interaction between XIAP/BIR3 and p10.

The above results were further confirmed using Far Western blot analysis with ³⁵S-labeled XIAP. Recombinant WT caspase-9, E306/D315/D330A, D315/330A, or D315A caspase-9 mutants, p12, p10, or Smac/DIABLO GST-fusion proteins were fractionated by SDS-PAGE and then blotted onto a nitrocellulose membrane using standard Western blotting technique. The proteins on the nitrocellulose membrane were denatured in a buffer (10 mM sodium phosphate pH 7.4, 150 mM sodium chloride, 5 mM magnesium chloride and 1 mM DTT) containing 6 M guanidine-HCl. These proteins were then renatured by gradual reduction of guanidine-HCl to 0.3 M. The membrane was blocked overnight in the same buffer containing 5% non-fat dry milk. The membrane was then probed with ³⁵S-labeled in vitro translated XIAP in the same buffer with 1% non-fat dry milk. The membrane was washed at least three times and then exposed to X-ray film.

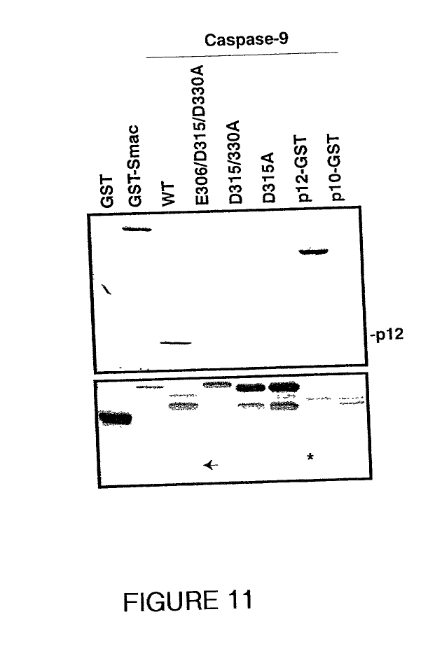

As shown in FIG. 11, XIAP was able to bind to the WT p12 subunit of caspase-9 (p12-GST) and Smac-GST bands. XIAP was not able to bind to variants of caspase-9 with Asp315 to Ala mutation, i.e., uncleavable caspase-9 (E306A/D315/D330A), caspase-9 D315A and caspase-9 D3 15/330A, GST, or caspase-9-p10 bands on the nitrocellulose filter. It should be noted that GST, uncleavable caspase-9 (E306A/D315/D330A), caspase-9 D315A, caspase-9 D315/330A, caspase-9-p35, and caspase-9-p10 all lacked an exposed BIR3-binding motif. The absence of interaction between p10 and XIAP by Far western suggested that the observed weak interaction (FIG. 11) was not physiologically significant. These results indicated that cleavage of caspase-9 at Asp315 exposed the XIAP-binding motif in the linker region, thereby allowing binding to the BIR3 domain of XIAP and concomitant inhibition of caspase-9 activity.

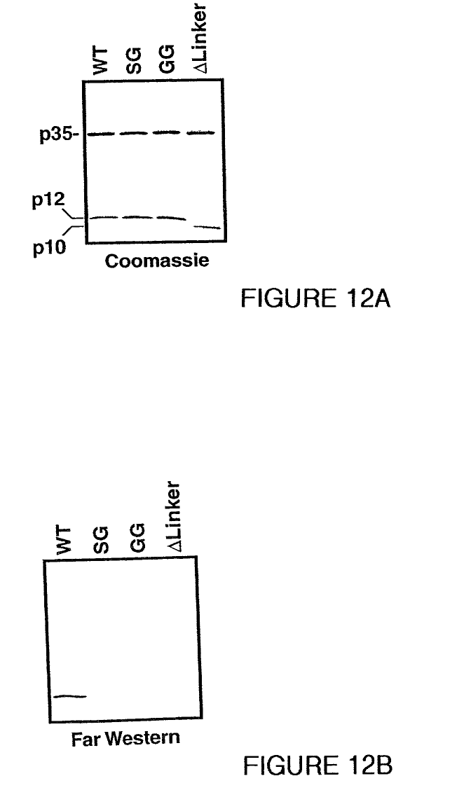

Since the BIR3 domain of XIAP is the domain that specifically targets caspase-9, these results suggested that the interaction between the linker region of caspase-9 and the BIR3 domain was primarily responsible for this inhibition. To determine the importance of the linker peptide/region for inhibition of caspase-9, residues 316 to 330 of SEQ ID NO:1 were deleted from caspase-9, and the deletion mutant was expressed in bacteria. The recombinant WT caspase-9 or Δlinker mutant was fractionated by SDS-PAGE and then Coomassie stained (see FIG. 12A) or analyzed by Far western as described above (see FIG. 12B). This deletion mutant (Δlinker) was able to undergo complete processing to generate the p35 and p10 subunits (FIG. 12A). As expected, the deletion mutant lost significantly the ability to interact with BIR3 (FIG. 12B).

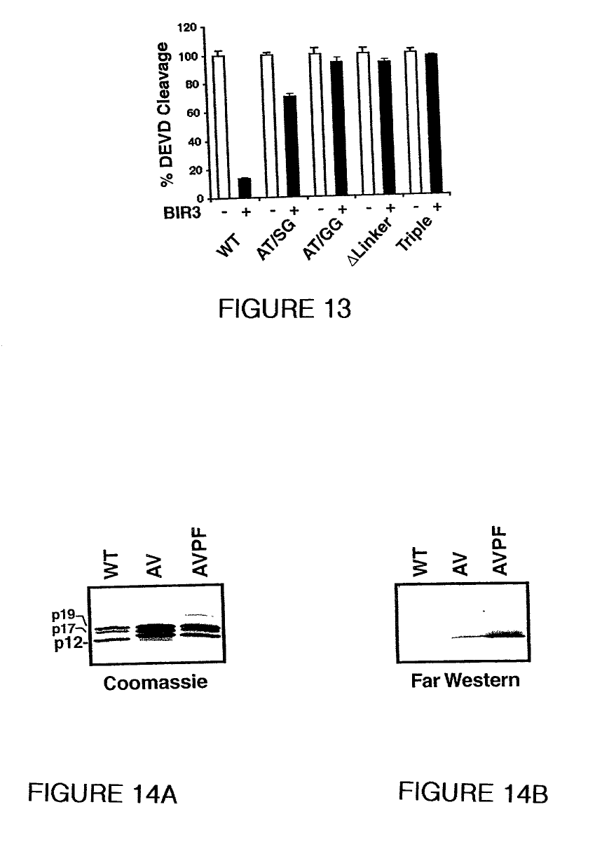

To test for enzymatic activity, caspase-9-depleted S100 extracts (20 μg) from Apaf-1-deficient mouse embryonic fibroblasts were incubated with recombinant Apaf-1, cytochrome c, dATP, and equal amounts (10 nM) of WT caspase-9 or the caspase-9 Δlinker mutant protein in the presence (+) or absence (−) of purified XIAP-BIR3 (20 nM). The reactions were carried out in the presence of the peptide substrate DEVD-AMC (100 μM) for 30 minutes, and substrate cleavage was measured by luminescence spectrometry. The data shown in FIG. 13 are represented in % activity relative to the DEVD-AMC cleaving activity in the absence of BIR3. As shown in FIG. 13, the deletion of the linker region did not inhibit enzymatic activity, even in the presence of XIAP-BIR3. This confirmed that the p10 subunit of caspase-9 was not the primary target of XIAP-inhibition and that the linker peptide was required for binding to BIR3 and for inhibition of caspase-9 activity.

To determine the importance of the first two residues of caspase-9-p12, these Ala-Thr residues were mutated to Ser—Gly or Gly—Gly. The recombinant WT caspase-9, caspase-9 AT316, 317SG or AT316, 317GG mutant proteins were fractionated by SDS-PAGE and then Coomassie stained (see FIG. 12A) or analyzed by Far western as described above (see FIG. 12B). The AT/SG or AT/GG mutants were completely processed at Asp315 to generate the p35 and p12 subunits (FIG. 12, left panel, lanes SG & GG). Like the linker-deletion mutant, both the SG and GG point mutant caspase-9 lost significantly the ability to interact with BIR3 (FIG. 12B). The data shown in FIG. 13 indicated that the first two residues in the p12 subunit of caspase-9 were important for binding to BIR3 and inhibition.

To test for enzymatic activity, caspase-9-depleted S100 extracts (20 μg) from Apaf-1-deficient mouse embryonic fibroblasts were incubated with recombinant Apaf-1, cytochrome c, dATP, and equal amounts (10 nM) of WT caspase-9, caspase-9 AT316, 317SG, or AT316, 317GG mutant proteins in the presence (+) or absence (−) of purified XIAP-BIR3 (20 nM). The reactions were carried out in the presence of the peptide substrate DEVD-AMC (100 μM) for 30 minutes, and substrate cleavage was measured by luminescence spectrometry. The data shown in FIG. 13 are represented in % activity relative to the DEVD-AMC cleaving activity in the absence of BIR3.

Since caspase-3 was not inhibited by BIR3 (IC₅₀>400 nM), it was examined whether substitution of the first four residues of caspase-3-p12 with AVPF could allow binding to and inhibition by BIR3. Recombinant WT caspase-3 or caspase-3 SG176, 177AV or SGVD176-179AVPF mutants were fractionated by SDS-PAGE and Coomassie stained (FIG. 14A) or analyzed by Far western as described above (FIG. 14B).

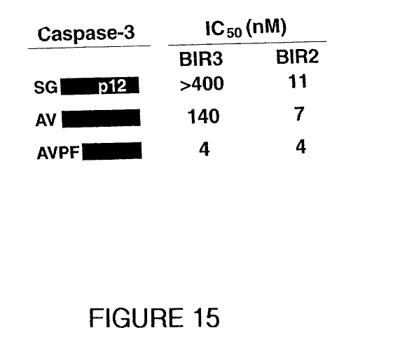

WT caspase-3 or caspase-3 SG176, 177AV or SGVD176-179AVPF mutant proteins (10 pM) were incubated with purified BIR3 (0.5-800 nM) or BIR1-BIR2 proteins (0.1-80 nM) at 37° C.) in the presence of DEVD-AMC (100 μM) for 30 minutes to determine the affects on the enzyme activity assays of caspase-3. The substrate cleavage was measured by luminescence spectrometry. The IC₅₀s were then calculated from the percentage of inhibition. As shown in FIG. 15, mutation of the first two residues of caspase-3 to Ala-Val allowed weak binding to XIAP and inhibition by BIR3 (IC₅₀˜140 nM). Mutation of the first four residues to AVPF enhanced binding to XIAP and increased inhibition by BIR3 (IC₅₀˜4 nM). These mutations also enhanced inhibition of caspase-3 by BIR2 of XIAP (IC₅₀s: WT˜10 nM, AV˜7 nM, AVPF˜4 nM). This is consistent with the recent findings that BIR2 could also bind the AVPI peptide of Smac/DIABLO (Chai, J. et al., Nature 406:855-862, 2000; Liu, Z. et al., Nature 408:1004-1008, 2000). Together, the above results clearly established that inhibition of human/mouse caspase-9 by XIAP was due to interaction of the ATPF/AVPY motif at the N-terminus of p12 with the BIR3 domain of XIAP.

### Example 4

### Binding of Caspase-9 or SMAC to IAPS is Mutually Exclusive

This example discloses that binding between Smac or the caspase-9-p12 and the BIR3 domain of IAPs is mutually exclusive.

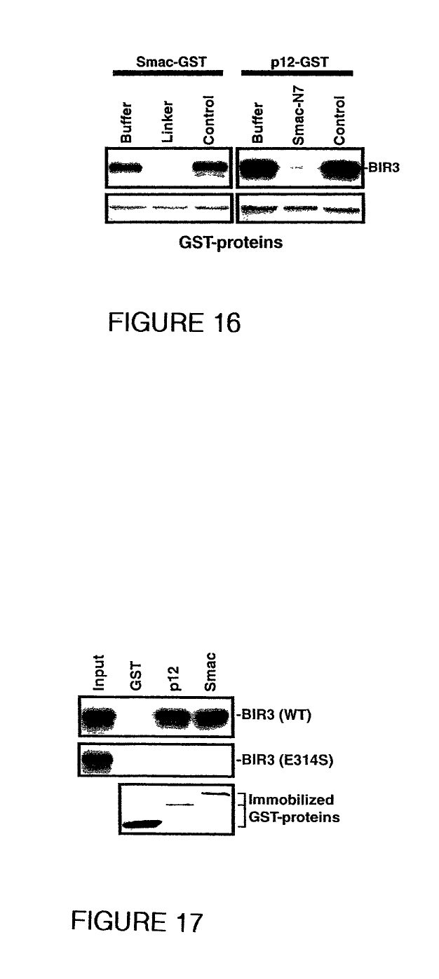

Since it is possible that Smac/DIABLO promotes caspase-9 activity by interfering with the interaction of the caspase-9-p 12 with the BIR3 domain of XIAP, it was determined if binding of caspase-9-p12 and Smac/DIABLO to the BIR3 domain was mutually exclusive. In vitro binding experiments were performed between Smac/DIABLO or caspase-9-p12 and BIR3 in the presence or absence of a chemically synthesized caspase-9 linker peptide (ATPFQEGLRTFDQLD, SEQ ID NO:11) or Smac-N7 peptide (AVPIAQK, SEQ ID NO:12), respectively. In a first in vitro binding experiment, Smac-GST was immobilized onto glutathione resin and then incubated with BIR3 in the absence of any peptide (FIG. 16, left panel, lane 1, buffer) or presence of 200 μM linker peptide (FIG. 16, left panel, lane 2, linker) or non-specific peptide (SEQ ID NO:14; FIG. 16, left panel, lane 3, control). In a second in vitro binding experiment, p12-GST was immobilized onto glutathione resin and then incubated with BIR3 in the absence of any peptide (FIG. 16, right panel, lane 1, buffer), or presence of 200 μM Smac-N7 peptide (FIG. 16, right panel, lane 2, Smac-N7) or a non-specific peptide (FIG. 16, right panel, lane 3, control). The interactions were analyzed as in Example 3.

As shown in FIG. 16, the linker peptide completely inhibited Smac/DIABLO binding to BIR3. Similarly, the Smac-N7 peptide completely inhibited binding of caspase-9-p12 to BIR3. The affinity of the linker peptide and the Smac-N7 peptide towards BIR3 were comparable (Linker, Kd 0.55±0.15 μM; Smac-N7, Kd ˜0.81±0.18 μM). Combined with the above data, this indicates that Smac/DIABLO competed with caspase-9 for binding to the same pocket on the surface of XIAP. This could explain the ability of Smac/DIABLO to promote the catalytic activity of caspase-9 in the presence of XIAP.

Next the interaction of caspase-9-p12 and mature Smac/DIABLO with WT and E314S mutant BIR3 domain of XIAP was examined. GST alone, GST-p12, or Smac/DIABLO were incubated with ³⁵S-labeled WT BIR3 or E314S mutant BIR3, and the interactions were analyzed as in Example 3. FIG. 17 shows that the mutation of a critical residue (E314), which was essential for binding to the Smac/DIABLO N-terminus and inhibition of caspase-9, abrogated binding of both Smac/DIABLO and caspase-9-p12 to BIR3.

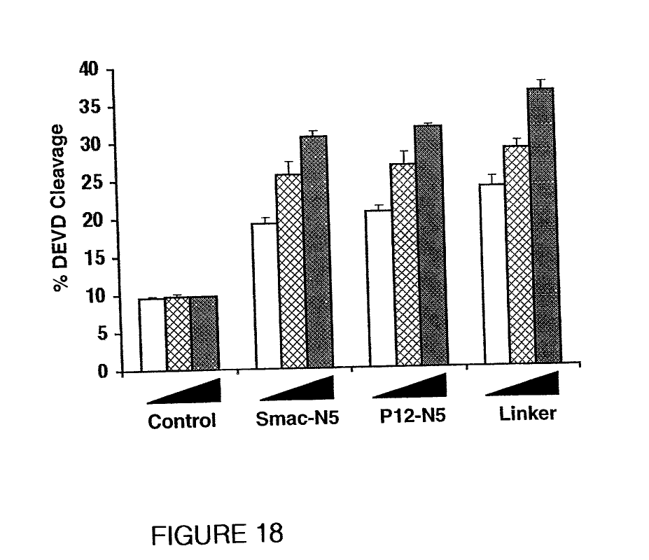

If the chemically synthesized linker peptide and processed caspase-9 bind to the same pocket on the surface of the BIR3 domain of XIAP, then it would be expected that the caspase-9 linker peptide should mimic the ability of Smac/DIABLO to promote caspase activation in S100 extracts in the presence of XIAP. To test this hypothesis, the ability of the caspase-9 linker peptide or a peptide containing only the first five residues of the caspase-9-p12 to promote cytochrome c-dependent activation of caspase-3 in S100 extracts containing XIAP was examined. The 293T S100 extracts were mixed with purified XIAP (10 nM) and then stimulated with cytochrome c and DATP in the presence of increasing amounts, 25, 100 and 500 μM, of a nonspecific peptide (control, MKSDFYFQK, SEQ ID NO:14), Smac-N5 (AVPIA, SEQ ID NO:20), p12-N5 (ATPFQ, SEQ ID NO:19) or linker (ATPFQEGLRTFDQLD, SEQ ID NO:11) peptides. The activity of caspase-3 in the S100 extracts was measured using the peptide substrate DEVD-AMC. Both the linker and the p12-N5 peptides were as effective in promoting caspase-3 activation as the Smac-N5 or N7 peptides in the XIAP containing extracts (FIG. 18). These results confirm that the linker peptide competed with caspase-9 for binding to BIR3 and functioned as an inhibitor of XIAP.

During apoptosis, caspase-9 is further processed at Asp330 by the activated caspase-3. Based on the above observations, processing at Asp330 not only relieved the inhibition of caspase-9 by XIAP, but also released the linker region into the cytoplasm, allowing it to bind to XIAP and neutralize its inhibitory activity. Thus, from a physiological aspect, the release of the linker peptide from caspase-9 during apoptosis constitutes a positive feedback loop in the potentiation of the caspase cascade and apoptosis.

### Example 5

### Proposed Models of Caspase-9 Linker Peptide, SMAC, or Caspase-9 Binding to the BIR3 Domain of XIAP

This example sets forth proposed models of caspase-9 linker peptide, Smac, or caspase-9 binding to the BIR3 domain of XIAP.

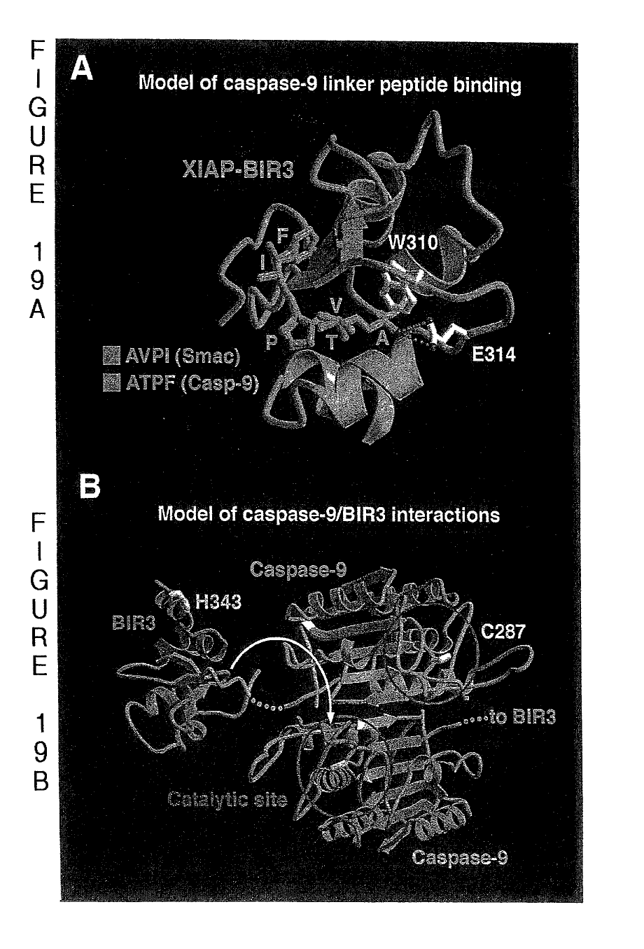

In the crystal structure of a Smac/DIABLO-XIAP complex, the N-terminal tetra-peptide of Smac/DIABLO binds a surface groove on the BIR3 domain of XIAP (FIG. 19A), making a network of hydrogen bond interactions and extensive van der Waals contacts. The side chain of the first residue Ala fits in a conserved hydrophobic pocket in the surface groove of XIAP, which is formed in part by Trp310 (FIG. 19A). The Ala main chain groups hydrogen bond to surrounding XIAP residues, including a pair of charge-stabilized hydrogen bonds to Glu314. The high sequence similarity between the N-terminal sequences of the p12 subunit of caspase-9 and Smac/DIABLO predicts an identical mode of interaction with the BIR3 domain of XIAP (FIG. 19A). This is indeed supported by experimental observations presented in the application. The first residue Ala of the tetra-peptide is partially embedded in a pocket and uses its fully exposed amino group to hydrogen bond to Glu314, explaining why procaspase-9 must be proteolytically processed before it can bind XIAP (FIG. 2). In agreement with this prediction, mutation of Trp310 or Glu314 resulted in abrogation or significant reduction of interactions with both Smac/DIABLO and caspase-9.

Clearly, the physical binding of the N-terminus of the caspase-9 p12 subunit to the BIR3 domain of XIAP constitutes an indispensable step in the inhibition of caspase-9. The close proximity of the N-terminus of the p12 subunit and the catalytic residue of caspase-9 suggests that XIAP may negatively affect entry of the substrate to the active site (FIG. 19B). This proposed model is further supported by the observation that mutation of His343 in XIAP-BIR3 resulted in complete loss of inhibition to the enzymatic activity of caspase-9, but not binding to caspase-9-p12. This indicates that His343 directly binds the active site of caspase-9. Thus, although binding of the tetra-peptide of caspase-9 by XIAP appears to be a major contributor in their mutual interaction, other weaker interactions between caspase-9 and XIAP also contributed to the inhibition of caspase-9. Furthermore, the IAP proteins are likely to dimerize in solution, which could further block substrate entry.

### Example 6

### Competivative Binding Assay

This example provides one example of a high throughput screen to identify organic or non-organic molecules that can disrupt the interaction of BIR-3 with the IAP-binding motif in caspase-9-p12 or Smac/DIABLO.

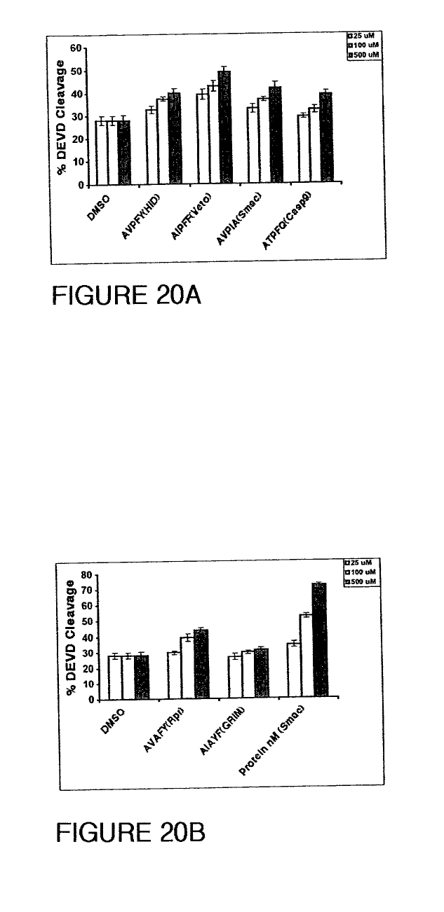

The purified caspase-3-AVPF mutant was mixed with XIAP-BIR3 (20 nM). This mixture was then incubated with increasing amounts, 25, 100 or 500 μM, of purified mature Smac or IAP-binding peptides derived from the N-termini of Hid (AVPFY, SEQ ID NO:23), Veto (AIPFF, SEQ ID NO:10), Smac (AVPIA, SEQ ID NO:24), caspase-9-p12 (ATPFQ, SEQ ID NO:25), Reaper (AVAFY, SEQ ID NO:26), or Grim (AIAYF, SEQ ID NO:27). The reactions were carried out in the presence of the peptide substrate DEVD-AMC (50 μM) for 30 minutes, and substrate cleavage was measured by luminescence spectrometry. The caspase activity in all the samples is plotted in FIG. 20 as a percentage of the activity of caspase-3 in the absence of XIAP-BIR3 (100%).

The above observations reveal an interesting mechanism for the activation and inhibition of caspase-9. Unlike other caspases, proteolytic processing of caspase-9 serves as a mechanism for inhibition, rather than activation. In the absence of proteolytic processing, XIAP is unable to interact with procaspase-9 or inhibit its enzymatic activity. Upon apoptotic stimuli, procaspase-9 undergoes auto-catalytic processing in the context of an Apaf-1 and cytochrome c-containing holoenzyme, in which the apoptosome serves as the allosteric regulator of the caspase-9 activity.

If Smac/DIABLO peptide interacts with the BIR3 domain of XIAP in the same manner as does caspase-9, how can Smac/DIABLO gain an edge in relieving the inhibition of capase-9? First, in addition to the tetra-peptide binding, Smac/DIABLO uses an extensive second interface to interact with the BIR3 domain of XIAP, involving over 2000 A² burial surface area. This additional interaction may tip the balance in favor of the Smac/DIABLO -XIAP complex. Second, Smac/DIABLO also binds tightly to the BIR2 domain of XIAP, which could facilitate the Smac-BIR3 interactions. Third, cleavage of caspase-9 after Asp330 releases the linker peptide, which further helps to remove the inhibition of caspase-9 by XIAP. Finally, in apoptotic cells, the amount of Smac released from the mitochondria could be in excess.

The activation of pro-caspase-9 represents a critical step in the mitochondria-initiated apoptotic pathways. Paradoxically, XIAP is unable to bind and inhibit procaspase-9 but binds and inhibits the proteolytically processed mature caspase-9. More strikingly, the mature caspase-9 uses the same conserved tetra-peptide to interact with XIAP as the mature form of Smac/DIABLO. These conserved interactions lead to opposing effects in caspase-9 activity and consequently apoptosis.

In providing the foregoing description of the invention, citation has been made to several references that will aid in the understanding or practice thereof. All such references are incorporated by reference herein.

From the foregoing it will be appreciated that, although specific embodiments of the invention have been described herein for purposes of illustration, various modifications may be made without deviating from the spirit and scope of the invention. In addition, all references including patents, patent applications, and journal articles are incorporated herein in their entirety. Accordingly, the invention is not limited except as by the appended claims.

1. An isolated nucleic acid molecule comprising a polynucleotide having a sequence encoding a peptide or polypeptide comprising at least an amino acid sequence set forth in SEQ ID NO:13, or a variant thereof, wherein said peptide or polypeptide specifically binds to at least a portion of an Inhibitor of Apoptosis Protein (IAP).

2. The isolated nucleic acid molecule of claim 1, wherein said portion is at least one BIR domain.

3. The isolated nucleic acid molecule of claim 2, wherein said BIR domain is Bir3.

4. The isolated nucleic acid molecule of claim 1, wherein said specific binding is to a full-length IAP.

5. The isolated nucleic acid molecule of claim 1, wherein said amino acid sequence is selected from the group consisting of the first four amino acid residues of each of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

6. An isolated nucleic acid molecule consisting essentially of a polynucleotide having a sequence encoding a peptide or polypeptide comprising at least an N-terminus amino acid sequence set forth in SEQ ID NO:11.

7. An isolated nucleic acid molecule consisting essentially of a polynucleotide having a sequence encoding a peptide or polypeptide comprising at least an N-terminus amino acid sequence of Ala—Val—Pro—Tyr, as set forth in SEQ ID NO:15.

8. An isolated nucleic acid molecule consisting essentially of a polynucleotide having a sequence encoding a peptide or polypeptide comprising at least an N-terminus amino acid sequence set forth in SEQ ID NO:12.

9. An isolated nucleic acid molecule comprising a polynucleotide having a sequence encoding a peptide or polypeptide comprising a first portion of a procaspase-9 that specifically binds at least a portion of an IAP and a second portion of a procaspase-9 containing a mutated active site, wherein said peptide or polypeptide specifically binds at least a portion of an IAP and lacks cysteine protease activity.

10. An isolated nucleic acid molecule comprising a polynucleotide having a sequence encoding a peptide or polypeptide comprising an amino acid sequence of SEQ ID NO:13, and further comprising at least a portion of a caspase-3, wherein said peptide or polypeptide exhibits caspase-3 enzymatic activity that is inhibited by an IAP or an IAP BIR3 domain.

11. The isolated nucleic acid molecule of claim 10, wherein the peptide or polypeptide consists essentially of a caspase-3 in which the amino acid residues corresponding to the amino-terminal two residues of the p12 subunit are substituted with Ala—Val.

12. The isolated nucleic acid molecule of claim 10, wherein the peptide or polypeptide consists essentially of a caspase-3 in which the amino acid residues corresponding to the amino-terminal four residues of the p12 subunit are substituted with residues set forth in SEQ ID NO:13.

13. An isolated nucleic acid molecule comprising a polynucleotide having a sequence encoding a peptide or polypeptide comprising at least a portion of a mutated procaspase-9, wherein said portion fails to undergo normal processing and said portion possesses wild type caspase-9 enzymatic activity.

14. The nucleic acid molecule of claim 13 wherein said portion of a mutated caspase-9 corresponds to SEQ ID NO:1 with an amino acid substitution, deletion, or addition.

15. The nucleic acid molecule of claim 13 wherein said portion of mutated procaspase-9 corresponds to SEQ ID NO:1 with amino acid residue 315 substituted by Ala.

16. The nucleic acid molecule of claim 13 wherein said portion of mutated procaspase-9 corresponds to SEQ ID NO:1 with amino acid residues 315 and 330 substituted by Ala.

17. The nucleic acid molecule of claim 13 wherein said portion of mutated procaspase-9 corresponds to SEQ ID NO:1 with amino acid residues 306, 315, and 330 substituted by Ala.

18. The nucleic acid molecule of claim 13 wherein said portion of mutated procaspase-9 corresponds to SEQ ID NO:1 with amino acid residues 316 through 330 deleted.

19. An expression vector comprising a nucleic acid molecule selected from the group consisting of claims 1-9 and 13-18, operatively linked to regulatory elements.

20. The expression vector of claim 19, wherein the regulatory elements include an inducible promoter.

21. A host cell containing the expression vector of claim 19.

22. The host cell of claim 21, wherein the cell is selected from the group consisting of a bacterium, a yeast, an animal cell, and a plant cell.

23. A peptide or polypeptide comprising at least an amino acid sequence set forth in SEQ ID NO:13, wherein said peptide or polypeptide specifically binds to at least a portion of an Inhibitor of Apoptosis Protein (IAP).

24. The peptide or polypeptide of claim 23, wherein said portion is at least one BIR domain.

25. The peptide or polypeptide of claim 23, wherein said BIR domain is BIR3.

26. The peptide or polypeptide of claim 23, wherein said specific binding is to a full-length IAP.

27. The peptide or polypeptide of claim 23, wherein said amino acid sequence is selected from the group consisting of the first four amino acid residues of each of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

28. A peptide or polypeptide comprising the amino acid residues set forth in SEQ ID NO:11 or a variant thereof, wherein said peptide or polypeptide specifically binds to at least a portion of an IAP.

29. A peptide or polypeptide comprising the amino acid residues set forth in SEQ ID NO:15 or a variant thereof, wherein said peptide or polypeptide specifically binds to at least a portion of an IAP.

30. A peptide or polypeptide comprising the amino acid residues set forth in SEQ ID NO:12 or a variant thereof, wherein said peptide or polypeptide specifically binds to at least a portion of an IAP.

31. A peptide or polypeptide comprising a first portion of a procaspase-9, or a variant thereof, that specifically binds at least a portion of an IAP and a second portion of a procaspase-9, or a variant thereof, containing a mutated active site, wherein said peptide or polypeptide specifically binds to at least a portion of an IAP and lacks cysteine protease activity.

32. A peptide or polypeptide comprising an amino acid sequence of SEQ ID NO:13, and further comprising at least a portion of a caspase-3, or a variant thereof, wherein said peptide or polypeptide exhibits caspase-3 enzymatic activity that is inhibited by an IAP BIR3 domain.

33. The peptide or polypeptide of claim 32 comprising a caspase-3, or a variant thereof, in which the amino acid residues corresponding to the amino-terminal two residues of the p12 subunit are substituted with Ala-Val.

34. The peptide or polypeptide of claim 32 comprising a caspase-3, or a variant thereof, in which the amino acid residues corresponding to the amino-terminal four residues of the p12 subunit are substituted with any four contiguous residues set forth in SEQ ID NO:13.

35. A peptide or polypeptide comprising at least a portion of a mutated procaspase-9 or a variant thereof, wherein said portion fails to undergo normal processing and said portion possesses wild type caspase-9 enzymatic activity.

36. The peptide or polypeptide of claim 35 wherein said portion of mutated procaspase-9 corresponds to SEQ ID NO:1 with amino acid residue 315 substituted by Ala.

37. The peptide or polypeptide of claim 35 wherein said portion of mutated procaspase-9 corresponds to SEQ ID NO:1 with amino acid residues 315 and 330 substituted by Ala's.

38. The peptide or polypeptide of claim 35 wherein said portion of mutated procaspase-9 corresponds to SEQ ID NO:1 with amino acid residues 306, 315, and 330 substituted by Ala's.

39. The peptide or polypeptide of claim 35 wherein said portion of mutated procaspase-9 corresponds to SEQ ID NO:1 with amino acid residues 316 through 330 deleted.

40. An antibody that specifically binds to a peptide or polypeptide set forth in SEQ ID NO:13 that specifically binds to at least a portion of an IAP.

41. The antibody of claim 40, wherein said antibody inhibits the binding of said peptide or polypeptide to said portion of an IAP.

42. An antibody that specifically binds to an epitope located on the N-terminus of a caspase-9-p12.

43. The antibody of claim 42, wherein said antibody inhibits the binding of a caspase-9-p12 to at least a portion of an IAP.

44. The antibody of claim 41 or 43, wherein said portion is at least one BIR domain.

45. The antibody of claim 44, wherein said BIR domain is BIR1.

46. The antibody of claim 44, wherein said BIR domain is BIR2.

47. The antibody of claim 44, wherein said BIR domain is BIR3.

48. The antibody of claim 42, wherein said antibody inhibits the binding to a full-length IAP.

49. A method for inducing apoptosis in a cell comprising contacting the cell with at least one component selected from the group consisting of:
(a) a peptide or polypeptide of claims 23-31 and 35-39;
(b) a nucleic acid molecule of claims 1-9 and 13-18; and
(c) an antibody of claims 40 and 42, under conditions and for a time sufficient to permit the induction of apoptosis in the cell.

50. The method of claim 49, wherein said peptide or polypeptide is capable of inhibiting caspase-9-p12 binding to at least a portion of an IAP.

51. The method of claim 50, wherein said portion is at least one BIR domain.

52. The method of claim 51, wherein said BIR domain is BIR3.

53. The method of claim 52, wherein said BIR domain is BIR1 or BIR2.

54. The method of claim 50, wherein said peptide or polypeptide inhibits binding to a full length IAP.

55. The method of claim 49, wherein said polypeptide is a procaspase-9 mutant that fails to undergo normal processing.

56. The method of claim 49, wherein said cell overexpresses a peptide or polypeptide capable of inhibiting IAP binding to caspase-9.

57. The method of claim 49, wherein said cell overexpresses a procaspase-9 mutant that fails to undergo normal processing.

58. A method of stimulating apoptosis in a neoplastic or tumor cell, comprising contacting the cell with at least one component selected from the group consisting of:
(a) a peptide or polypeptide of claims 23 -31 and 35-39 and
(b) a nucleic acid molecule of claims 1-9 and 13-18; and
(c) an antibody of claims 40 and 42,
under conditions and for a time sufficient to permit the induction of apoptosis in the cell.

59. The method of claim 58, wherein said peptide or polypeptide is capable of inhibiting caspase-9-p12 binding to at least a portion of an IAP.

60. The method of claim 58, wherein said peptide or polypeptide is a procaspase-9 mutant that fails to undergo normal processing.

61. The method of claim 58, wherein said cell overexpresses a peptide of polypeptide capable of inhibiting caspase-9-p 12 binding to at least a portion of an IAP.

62. The method of claim 58, wherein said cell overexpresses a procaspase-9 mutant that fails to undergo normal processing.

63. The method of claim 58, wherein said cell overexpresses an inhibitor of a caspase.

64. The method of claim 63, wherein the inhibitor inhibits activation or activity of caspase-9.

65. The method of claim 63, wherein the inhibitor is at least a portion of an Inhibitor of Apoptosis protein.

66. A method of identifying an inhibitor or enhancer of a caspase-mediated apoptosis comprising:
(a) contacting a cell containing a vector expressing a peptide or polypeptide comprising at least an amino acid sequence set forth in SEQ ID NO:13 that is capable of specifically binding to at least a portion of an IAP with a candidate inhibitor or candidate enhancer; and
(b) detecting cell viability,
wherein an increase in cell viability as compared to a control indicates the presence of an inhibitor and a decrease in cell viability as compared to a control indicates the presence of an enhancer.

67. A method of identifying an inhibitor or enhancer of a caspase-mediated apoptosis comprising:
(a) contacting a cell containing a vector expressing a polypeptide selected from the group consisting of the polypeptides of claims 35-39; and
(b) detecting cell viability,
wherein an increase in cell viability as compared to a control indicates the presence of an inhibitor and a decrease in cell viability as compared to a control indicates the presence of an enhancer.

68. A method of identifying an inhibitor or enhancer of a caspase-mediated apoptosis comprising:
(a) contacting a cell containing a vector expressing a peptide or polypeptide comprising at least an amino acid sequence corresponding to SEQ ID NO:13 that is capable of specifically binding to at least a portion of an IAP with a candidate inhibitor or candidate enhancer; and
(b) detecting the presence of large and small caspase subunits, and therefrom determining the level of caspase processing activity,
wherein a decrease in processing as compared to a control indicates the presence of an inhibitor and an increase in processing indicates the presence of an enhancer.

69. The method of claim 68, wherein the caspase detected is selected from the group consisting of caspase-3, caspase-7 and caspase-9.

70. A method of identifying an inhibitor or enhancer of a caspase-mediated apoptosis comprising:
(a) contacting a cell containing a vector expressing a polypeptide selected from the group consisting of the polypeptides of claims 35-39; and
(b) detecting the presence of large and small caspase subunits, and therefrom determining the level of caspase processing activity,
wherein a decrease in processing as compared to a control indicates the presence of an inhibitor and an increase in processing indicates the presence of an enhancer.

71. The method of claim 70, wherein the caspase detected is selected from the group consisting of caspase-3, caspase-7 and caspase-9.

72. A method of identifying an inhibitor or enhancer of a caspase-mediated apoptosis comprising:
(a) contacting a cell containing a vector expressing a peptide or polypeptide comprising at least an amino acid sequence corresponding to SEQ ID NO:13 that is capable of specifically binding to at least a portion of an IAP with a candidate inhibitor or candidate enhancer; and
(b) detecting caspase enzymatic activity,
wherein a decrease in enzymatic activity as compared to a control indicates the presence of an inhibitor and an increase in enzymatic activity indicates the presence of an enhancer.

73. The method of claim 72, wherein the caspase enzymatic activity detected is selected from the group consisting of caspase-3, caspase-7 and caspase-9.

74. The method of claim 72, wherein the caspase enzymatic activity detected is a presence of a substrate cleavage product produced by a caspase cleavage of a substrate.

75. The method of claim 65, wherein said substrate is acetyl DEVD-aminomethyl coumarin.

76. A method of identifying an inhibitor or enhancer of a caspase-mediated apoptosis comprising:
(a) contacting a cell containing a vector expressing a polypeptide selected from the group consisting of the polypeptides of claims 35 -39 with a candidate inhibitor or enhancer; and
(b) detecting caspase enzymatic activity,
wherein a decrease in enzymatic activity as compared to a control indicates the presence of an inhibitor and an increase in enzymatic activity indicates the presence of an enhancer.

77. The method of claim 76, wherein the caspase enzymatic activity detected is selected from the group consisting of caspase-3, caspase-7 and caspase-9.

78. The method of claim 76, wherein the caspase enzymatic activity detected is a presence of a substrate cleavage product produced by a caspase cleavage of a substrate.

79. The method of claim 78, wherein said substrate is acetyl DEVD-aminomethyl coumarin.

80. A method for identifying a compound that inhibits a peptide or polypeptide comprising an amino acid sequence set forth in SEQ ID NO:13 that specifically binds at least a portion of an IAP from binding to said portion of an IAP, comprising:
(a) contacting a candidate compound with said peptide or polypeptide in the presence of said portion of an IAP; and
(b) detecting displacement or inhibition of binding of said portion of an IAP from said peptide or polypeptide.

81. The method of claim 80, wherein said portion of an IAP is a BIR3 domain.

82. The method of claim 80, wherein said portion of an IAP is a full length IAP.

83. A method for identifying a compound that inhibits a peptide or polypeptide comprising an amino acid sequence set forth in SEQ ID NO:13 that specifically binds at least a portion of an IAP from binding to said portion of an IAP, comprising:
(a) contacting a candidate compound with said peptide or polypeptide in the presence of said portion of an IAP; and
(b) performing a functional assay that confirms displacement of said portion of an IAP from said peptide or polypeptide.

84. The method of claim 83, wherein the functional assay detects the presence of large and small caspase subunits, and therefrom determining the level of caspase processing activity, wherein a decrease in processing confirms displacement.

85. The method of claim 84, wherein the caspase detected is selected from the group consisting of caspase-3, caspase-7 and caspase-9.

86. The method of claim 83, wherein the flnctional assay detects the presence of a substrate cleavage product produced by a caspase cleavage of a substrate.

87. The method of claim 86, wherein said substrate is acetyl DEVD-aminomethyl coumarin.

88. A composition comprising a nucleic acid molecule selected from the group consisting of claims 1-9 and 13-18, and a physiologically acceptable carrier.

89. A composition comprising the expression vector of claim 19, and a physiologically acceptable carrier.

90. A composition comprising a peptide selected from the group consisting of claims 23-31 and 35-39, and a physiologically acceptable carrier.

91. A composition comprising an antibody of claim 40 or 42, and a physiologically acceptable carrier.

92. A composition comprising an inhibitor or enhancer of apoptosis identified by a method selected from the group consisting of claims 57-59, 61, 63, 67, and 71.

93. A method of producing a compound for inhibiting or enhancing apoptosis in a cell, comprising:
(a) identifying an inhibitor or enhancer of apoptosis according to a method selected from the group consisting of claims 66-68, 70, 72, 76, and 80; and
(b) purifying said inhibitor or enhancer.

94. A process for the manufacture of a compound for inhibiting or enhancing apoptosis in a cell, comprising:
(a) identifying an inhibitor or enhancer of apoptosis according to a method selected from the group consisting of claims 66-68, 70, 72, 76, and 80; and
(b) derivitizing the compound of (a) and optionally repeating at least one of steps (a) and (b),
to produce a compound that inhibits or enhances apoptosis.


SEQUENCE LISTING (28 sequences)

SEQ ID NO: 1 - protein, 416 aa, Homo sapiens
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 2 - protein, 15 aa, Drosophila sp.
AVAFYIPDQATLLRE

SEQ ID NO: 3 - protein, 15 aa, Drosophila sp.
AIAYFIPDQAQLLAR

SEQ ID NO: 4 - protein, 15 aa, Drosophila sp.
AVPFYLPEGGADDVA

SEQ ID NO: 5 - protein, 15 aa, Mus musculus
AVPYQEGPRPLDQLD

SEQ ID NO: 6 - protein, 15 aa, Homo sapiens
ATPFQEGLRTFDQLD

SEQ ID NO: 7 - protein, 15 aa, Xenopus sp.
ATPVFSGEGDRDEVD

SEQ ID NO: 8 - protein, 15 aa, Homo sapiens
AVPIAQKSEPHSLSN

SEQ ID NO: 9 - protein, 5 aa, Homo sapeins
AVPSP

SEQ ID NO: 10 - protein, 5 aa, Homo sapiens
AIPFF

SEQ ID NO: 11 - protein, 15 aa, Homo sapiens
ATPFQEGLRTFDQLD

SEQ ID NO: 12 - protein, 7 aa, Homo sapiens
AVPIAQK

SEQ ID NO: 13 - protein, 4 aa, Artificial Sequence
  Consensus IAP-binding motif
AXXX

SEQ ID NO: 14 - protein, 9 aa, Artificial Sequence
  Non-specific peptide
MKSDFYFQK

SEQ ID NO: 15 - protein, 4 aa, Mus musculus
AVPY

SEQ ID NO: 16 - DNA, 1480 bp, Homo sapiens
gccatggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataaggccagggcccctcaccctgccttatcttgcaccccaaagctttcctgccccaggcctgaaagaggctgaggcctggactttcctgcaactcaaggactttgcagccggcacagggtctgctctttctctgccagtgacagacaggctcttagcagcttccagattgacgacaagtgctgaacagtggaggaagagggacagatgaatgccgtggattgcacgtgg

SEQ ID NO: 17 - protein, 5 aa, Artificial Sequence
  Consensus cysteine protease active site.
QACXG

SEQ ID NO: 18 - protein, 32 aa, Homo sapiens
PEDESPGSNPEPDATPFQEGLRTFDQLDAISS

SEQ ID NO: 19 - protein, 5 aa, Homo sapiens
ATPFQ

SEQ ID NO: 20 - protein, 5 aa, Homo sapiens
AVPIA

SEQ ID NO: 21 - protein, 4 aa, Homo sapiens
AVPI

SEQ ID NO: 22 - protein, 32 aa, Homo sapiens; identical to SEQ ID NO: 18

SEQ ID NO: 23 - protein, 5 aa, Drosophila sp.
AVPFY

SEQ ID NO: 24 - protein, 5 aa, Homo sapiens
AVPIA

SEQ ID NO: 25 - protein, 5 aa, Homo sapiens
ATPFQ

SEQ ID NO: 26 - protein, 5 aa, Drosophila sp.
AVAFY

SEQ ID NO: 27 - protein, 5 aa, Drosophila sp.
AIAYF

SEQ ID NO: 28 - protein, 4 aa, Homo sapiens
ATPF